(19) United States
(12) Patent Application Publication
(10) Pub. No.: US 2026/0262684 A1
(43) Pub. Date: Sep. 10, 2026

(54) FAST HYDRATION FORMULATION AND METHODS

(71) Applicant: THE COCA-COLA COMPANY, Atlanta, GA (US)

(72) Inventors: Haiyu REN, Atlanta, GA (US); Yu SHI, Atlanta, GA (US); Nilesh Bansilal KARAVA, Atlanta, GA (US); Dattatreya GAJULA, Atlanta, GA (US); HyunGyu SUH, Atlanta, GA (US)

(21) Appl. No.: 18/870,139

(22) PCT Filed: May 30, 2023
(86) PCT No.: PCT/US2023/067625

§ 371 Date: Nov. 27, 2024

Related U.S. Application Data
(60) Provisional application No. 63/346,470, filed on May 27, 2022.

(51) Int. Cl. A23L 2/60; A23L 2/395; A23L 33/16; A23L 33/20

(52) U.S. Cl. CPC A23L 2/60; A23L 2/395; A23L 33/16; A23L 33/20

(57) ABSTRACT

The present disclosure presents compositions and methods for fast hydration, fast rehydration, long lasting hydration, and/or long lasting rehydration. A composition for oral consumption comprises at least one active ingredient, and in some embodiments the composition is substantially free of nutritive sweetener. A method of fast hydrating, fast rehydrating, providing long lasting hydration, and/or providing long lasting rehydration in a human, the method comprising administering an effective amount of an oral composition, wherein the oral composition comprises at least one active ingredient, and in some embodiment the composition is substantially free of nutritive sweetener.

This application is being filed on May 30, 2023, as a PCT International Patent application and claims the benefit of and priority to U.S. Provisional patent application Ser. No. 63/346,470, filed May 27, 2022, the entire disclosure of which is incorporated by reference herein in its entirety.

### INTRODUCTION

The present disclosure generally relates to compositions and methods for fast hydration, fast rehydration, long lasting hydration, and/or long lasting rehydration.

Humans often suffer from dehydration or hypohydration under stressful conditions such as exposure to excess heat, heavy or extended periods of exercise, demanding physical activity with limited water, high-mountain climbing, or during extended water loss from diarrhea, vomiting or sweating. Negative physiological effects such as loss of electrolytes balances or proteins are often accompanied with dehydration. It would be beneficial to provide a composition for rapid and effective hydration or rehydration, which could quickly restore body water and critical elements to avoid any potential adverse health issues with dehydration.

Attempts have been made previously to prepare rehydration compositions and in particular, beverages and sport drinks, which provide the energy source, electrolytes, and water for rehydration. These beverages may contain mixtures of proteins, sugars such as glucose, fructose, maltose, salts, and other additives such as citric acid, glycerol, triacylgylcerol, sodium acid sulfate, which are alleged to be beneficial for rehydration. These beverages and compositions are generally disclosed, for examples, in U.S. Pat. Nos. 4,853,237, 5,447,730, 6,221,910, 6,485,764, 7,001,612, 7,160,565, 8,993,032, and U.S. Pat. App. Nos. 2005/0100637, 2005/0048136, 2009/0117224, 2012/0128815.

However, in any disclosed compositions could be more effective. For example, many compositions do not provide rapid restoration of body fluid and plasma D₂0 percent, and the duration for rehydration effect is often relatively short. In particular, the retention of body fluid after ingesting these compositions may not last long, and the total body water decreases quickly to the level prior to rehydration (Jeukendrup, Nutr. Metab, 2009). Additionally, the effective component of the rehyrdrating composition and/or the direct evidence of rehydration effect are not clearly indicated in prior disclosures. Moreover, many existing compositions are high in carbohydrates, and/or other high calorie ingredients that may be less preferred by consumers. There is also trade-off between the goal of rapid fluid restoration and the goal of energy supplementation when a person consumes a carbohydrate-containing drink. As the carbohydrate concentration of a drink increases, its energy density and osmolality increases, thereby reducing its gastric emptying rate Consequently, the effectiveness of fluid restoration is undermined.

Therefore, in spite of the above disclosures, it is still highly desirable for new fast hydrating, fast rehydrating, long lasting hydration, and/or long lasting rehydration compositions and beverages that provide a fast-acting and effective solution to dehydration and hypohydration, long duration of hydration or rehydration, compensation and extended period of retention of total body water, extended restoration of electrolyte balance, and/or improved taste and palatability profile.

### SUMMARY OF DISCLOSURE

The present disclosure presents fast hydrating, fast rehydrating, long lasting hydration, and/or long lasting rehydration compositions and methods that meet the above stated needs.

In one aspect, the present disclosure relates to a composition for oral consumption comprising at least one or at least two active ingredients in some embodiments, the composition can be an amino acid free formulation and/or a composition substantially free of nutritive sweetener. In some embodiments, the oral composition is a ready-to-drink hydrating beverage. One particular example is a sport beverage or sport drink. In some embodiments, the beverage has a concentration of active ingredient from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, based on the total volume of the beverage. In some embodiments, the oral composition has a concentration of at least one active ingredient comprising a mixture of sodium salts from about 15 mmol/L and about 35 mmol/L, or about between 17 mmol/L and about 30 mmol/L, or between about 20 mmol/L and about 30 mmol/L, or between about 23 mmol/L and about 28 mmol/L, or between about 25 mmol/L and about 26 mmol/L, or about 25.9 mmol/L. In some embodiments, the oral composition has a concentration of at least one active ingredient comprising a mixture of potassium salts from about 15 mmol/L and about 35 mmol/L, or about between 17 mmol/L and about 30 mmol/L, or between about 23 mmol/L and about 29 mmol/L, or between about 25 mmol/L and about 28 mmol/L, or about 27.8 mmol/L. In certain embodiments, the at least two active ingredients are a first active ingredient of sodium salts having a concentration between about 10 mmol/L and about 28 mmol/L, or about between 13 mmol/L and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.

In some embodiments, the oral composition is in a dry or semi-dry form. One particular example is a dry powder that is readily soluble in water. In some embodiments, a drinkable solution of the dry powder can be readily prepared by dissolving the dry powder in a drinkable medium, wherein the solution has a concentration that is free from amino acids. In some embodiments, the present composition further comprises at least one electrolyte. Examples of electrolyte include sodium, potassium, calcium, magnesium, chloride, phosphate, bicarbonate, and combinations thereof.

In some embodiments, the present composition further comprises at least one sweetener. The sweetener can be a carbohydrate, a peptide-based sweetener, a non-nutritive sweetener, an artificial sweetener, a commercial sweetener or sweetening composition, or a “natural high potency sweetener” (NHPS). In one particular embodiment, the present composition comprises a low- or non-calorie sweetener. In another particular embodiment, the present composition is free or substantially free from a non-nutritive sweetener and/or nutritive sweetener.

In some embodiments, the present composition further include at least one additive, at least one functional ingredient, or both.

In some embodiments, the present composition has an osmolality from about 250 to about 350 mOsm/kg, or from about 260 to about 340 mOsm/kg, or from about 270 to about 330 mOsm/kg or from about 280 to about 320 mOsm/kg, or from about 290 to about 310 mOsm/kg, or from about 290 to about 300 mOsm/kg.

In another aspect, the present disclosure relates to a method for hydration or rehydration. The present method utilizes the compositions described herein for at least one of the following purposes: providing a rapid impact on total body water restoration during rehydration, attenuating or reversing the effects of dehydration or hypohydration, ameliorating other adverse effects of exercise, heat or other activity which causes bodily fluid loss, providing a positive impact on subsequent physical performance, enhancing the duration of body fluid retention, rapidly increasing total body water, maintaining the increased total body water for long duration, restoring and maintaining electrolyte balance, providing energy source, balancing or controlling calorie uptake, and stimulating thirst and drinking.

In some embodiments, the present disclosure provides a method of fast hydrating, fast rehydrating, provide long lasting hydration, and/or provide long lasting rehydration to a human, the method comprising administering an effective amount of the oral composition described herein. In some embodiments of the present method, the oral composition is in a dry or semi dry form. The form of the oral composition may be beverage concentrate, gel, dry powder, tablet, or capsule. In one embodiment, the oral composition is in dry powder that is readily soluble in a drinkable medium. In some embodiments, the method include preparing a drinkable solution containing the dry powder by dissolving the dry powder in a drinkable medium comprising water, and administering the drinkable solution orally.

In other embodiments, the method include ingesting/consuming the oral composition and a drinkable medium comprising water. Ingestion of the oral composition and a drinkable medium can be concurrently, simultaneously, separately, or successively. In some embodiments, the ratio of the oral composition to the drinkable medium is such that the content of the active ingredients in the formulation is from about 1 g/L to about 50 g/L, or from about 2 g/L, to about 30 g/L, or from about 3 g/L to about 30 g/L, or from about 4 g/L to about 20 g/L, or from about 5 g/L to about 10 g/L, or from about 0.05 g/L to about 1 g/L, relative to the total volume of the drinkable medium. In some embodiments, the oral composition has a concentration of at least one active ingredient comprising a mixture of sodium salts from about 15 mmol/L and about 35 mmol/L, or about between 17 mmol/L and about 30 mmol/L, or between about 20 mmol/L and about 30 mmol/L, or between about 23 mmol/L and about 28 mmol/L, or between about 25 mmol/L and about 26 mmol/L, or about 25.9 mmol/L. In some embodiments, the oral composition has a concentration of at least one active ingredient comprising a mixture of potassium salts from about 15-mmol/L and about 35 mmol/L, or about between 17 mmol/L and about 30 mmol/L, or between about 23 mmol/L and about 29 mmol/L, or between about 25 mmol/L and about 28 mmol/L, or about 27.8 mmol/L. In certain embodiments, the at least two active ingredients are a first active ingredient of sodium salts having a concentration between about 10 mmol/L and about 28 mmol/L, or about between 13 mmol/L and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.

In some embodiments of the present method, the plasma volume of the human is increased by at least about 2.5%, or at least about 3%, or at least about 4% on or before 10 minutes after administration of the oral composition.

In some embodiments, the plasma D₂O percent of the human is increased by at least about 4.5%, or at least about 5%, or at least about 5.5%, or at least about 6% on or before 15 minutes after administration of the oral composition.

In some embodiments, the plasma D₂O percent of the human is increased by at least about 5%, or at least about 5.5%, or at least about 6%, or at least about 6.5%, or at least about 7% on or before 30 minutes after administration of the oral composition.

In some embodiments, the plasma D₂O percent of the human is increased by at least about 3%, or at least about 3.5%, or at least about 4%, or at least about 5%, or at least about 6% on or before 45 minutes after administration of the oral composition.

In some embodiments, the plasma D₂0 percent of the human is increased by at least about 3%, or at least about 3.5%, or at least about 4%, or at least about 5%, or at least about 6% on or before 60 minutes after administration of the oral composition.

In some embodiments, the plasma D₂O percent of the human measured at 30 minutes after administration of the oral composition remains substantially unchanged thereafter for at least about 15 minutes, or at least about 30 minutes, or at least about 1 hour.

In some embodiments, the plasma D₂O percent of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.

In some embodiments of the present method, the plasma osmolality of the human is maintained in a range from about 270 to about 330 mOsm/kg, or from about 280 to about 320 mOsm/kg, or from about 290 to about 31.0 mOsm/k, or from about 290 to about 300 mOsm/kg, in at least 60 minutes after administration of the oral composition.

In some embodiments, the change of the plasma osmolality of the human is no greater than 3 mOsm/kg in at least 60 minutes after administration of the oral composition.

### Definition and Interpretation of Selected Terms

As used herein, “weight percent,” “wt %, “percent by weight,” “% by weight,” and variations thereof refer to the concentration of a substance as the weight of that substance divided by the total weight of the composition and multiplied by 100. It is understood that, as used here, “percent,” “%,” and the like are intended to be synonymous with “weight percent,” “wt %,” etc.

As used herein, “g” represents gram; “kg” represents kilogram or 1000 grams; “L” represents liter; “mg” represents “milligram (10⁻³ gram);” “mL” or “cc” represents milliliter (10⁻³ liter). The units “g/100 g,” “g/100 mL,” or “g/L” are units of concentration or content of a component in a composition. One “mg/L” equals to one ppm (part per million). “Da” refers to Dalton, which is the unit for molecular weight; One Da equals to one g/mol. The unit of temperature used herein is degree Celsius (° C.).

The term “about” is used in conjunction with numeric values to include normal variations in measurements as expected by persons skilled in the art, and is understood to have the same meaning as “approximately” and to cover a typical margin of error, such as +15%, ±10%, ±5%, 1%, ±0.5%, or even 0.1% of the stated value. The term “about” also encompasses amounts that differ due to different equilibrium conditions for a composition resulting from a particular initial composition. Whether or not modified by the term. “about,” the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise. As used herein, “and/or” refers to and encompasses any and all possible combinations of one or more of the associated listed items, as well as the lack of combinations when interpreted in the alternative (“or”).

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5, 1-4; 1-3; 1-2; 2-5; 2-4; 2-3, 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “substantially free” may refer to any component that the composition of the disclosure lacks or mostly lacks. When referring to “substantially free” it is intended that the component is not intentionally added to compositions of the disclosure. Use of the term “substantially free” of a component allows for trace amounts of that component to be included in compositions of the disclosure because they are present in another component. However, it is recognized that only trace or de minimus amounts of a component will be allowed when the composition is said to be “substantially free” of that component. Moreover, if a composition is said to be “substantially free” of a component, if the component is present in trace or de minimus amounts it is understood that it will not affect the effectiveness of the composition. It is understood that if an ingredient is not expressly included herein or its possible inclusion is not stated herein, the disclosure composition may be substantially free of that ingredient. Likewise, the express inclusion of an ingredient allows for its express exclusion thereby allo-wing a composition to be substantially free of that expressly stated ingredient.

The term “comprise,” “comprises,” and “comprising” as used herein, specify the presence of the stated features, integers, steps, operations, elements, and/or components, but do not preclude the presence or addition of one or more other features, integers, steps, operations, elements, components, and/orgroups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising”.

As used herein, the terms “increase,” “increasing” “increased,” “enhance,” “enhanced,” “enhancing,” and “enhancement” (and grammatical variations thereof) describe an elevation of at least about 1%, 5%, 10%, 15%, 25%, 50%, 75%, 100%, 150%, 200%, 300%, 400%, 500% or more as compared to a control.

As used herein, the terms “reduce,” “reduced,” “reducing,” “reduction.” “diminish,” and “decrease” (and grammatical variations thereof), describe, for example, a decrease of at least about 1%, 5%, 10%, 15%, 20%, 25%, 35%, 50%, 75%, 80%, 85%, 90% 95%, 97%, 98%, 99%, or 100% as compared to a control. In particular embodiments, the reduction can result in no or essentially no (i.e., an insignificant amount, e.g., less than about 10% or even 5% or even 1%) detectable activity or amount.

“Osmolality” is defined as the number of dissolved particles in a unit volume of water solution. Osmolarity is defined as the number of dissolved particles in a unit weight of water solution. As a practical matter, osmolality and osmolarity have numerical values which are very close in the ranges involved in the present disclosure and therefore are used interchangeably. A solution that has 1/1000 of an osmol dissolved per kilogram has a concentration of 1 milliosmols (“mOs”) per kilogram. An osmol is the number of particles in 1 gram molecular weight of undissociated solute. Tonicity is a measure of the osmotic pressure of a solution relative to the osmotic pressure of the blood fluids. It is to be understood that the osmotic pressure of the body varies somewhat from one person to the other person. A hypotonic solution is a solution of lower osmotic pressure or tonicity than that of blood. The osmolality of a hypotonic solution is usually in the range of about 80-250 mOs/kg. An isotonic solution has the same tonicity as blood. Here, the osmolality usually ranges from about 280 to about 310 mOs/kg. A hypertonic solution is a solution of greater tonicity than blood. It normally has an osmolality range of about 310-440 mOs/kg. W ater has the osmolality of about 10-20 mOs/kg.

The term “beverage” as used herein means any drinkable liquid or semi-liquid, including for example water, flavored water, soft drinks, fruit drinks, tea-based drinks, juice-based drinks, gel drinks, carbonated or non-carbonated drinks, and alcoholic or non-alcoholic drinks. In some embodiments, a beverage powder may first be mixed with any drinkable liquid or semi-liquid to obtain a beverage.

As used herein dehydration is defined as a condition that occurs when the body loses too much water and other fluids that it needs to work normally. Dehydration is usually caused by severe diarrhea and vomiting, but it may also be caused by not drinking enough water or other fluids, sweating too much, fever, urinating too much, taking certain medicines, or physical exertion. Rehydration is the replenishment of water and electrolytes lost through dehydration. Fast rehydration (or fast hydration) is the replenishment of water and electrolytes within 30 minutes.

As used herein, “amino acid” refers to an organic compound or unit that contains amino (—NH₂) and carboxyl (—COOH) functional groups. The “amino acid” of the present disclosure broadly encompasses any compound having at least one amino acid unit. The “amino acid-free formulation” used herein refers to a formulation that is free from a molecule, a compound, a complex, an oligomer, a polymer, a mixture, or a composition having at least one amino acid unit physically (through non-covenant bonding) or chemically (though covalent, hydrogen, or coordinate bonding) incorporated in the formulation. Non-limiting examples of amino acid, amino acid compound, and amino acid used herein include aspartic acid, alanine, glycine, glutamic acid, praline, threonine, theanine, cysteine, cystine, alanine, valine, tyrosine, leucine, arabinose, trans-4-hydroxyproline, isoleucine, asparagine, serine, lysine, histidine, ornithine, methionine, carnitine, aminobutyric acid (α-, β-, and/or γ-isomers), glutamine, hydroxyproline, taurine, norvaline, sarcosine, and their salt forms such as sodium or potassium salts or acid salts. The amino acid also may be in the D- or L-configuration and in the mono-, di-, or tri-form of the same or different amino acids. Additionally, the amino acids may be α-, β-, and/or γ-isomers if appropriate. Combinations of the foregoing amino acids and their corresponding, salts (e.g. sodium, potassium, calcium, magnesium salts or other alkali or alkaline earth metal salts thereof, or acid salts) also are suitable additives in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, modified amino acids encompass both modified and unmodified amino acids. As used herein, amino acids also encompass both peptides, oligopeptides, and polypeptides (e.g., dipeptides, tripeptides, tetrapeptides, and pentapeptides) such as glutathione and L-alanyl-L-glutamine. Suitable polyamino acid include poly-L-aspartic acid, poly-L-lysine (e.g., poly-L-α-lysine or poly-L-ε-lysine), poly-L-ornithine (e.g., poly-L-α-ornithine or poly-L-εornithine), poly-L-alanine, other polymeric forms of amino acids, and salt forms thereof (e.g., calcium, potassium, sodium, or magnesium salts such as L-glutamic acid mono sodium salt). The poly-amino acid additives also may be in the D- or L-configuration. Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts or other alkali or alkaline earth metal salts thereof or acid salts) also are suitable additives in some embodiments. The poly-amino acids described herein also may comprise co-polymers of different amino acids. The poly-amino acids described herein also may comprise co-polymers of different amino acids. The poly-amino acids may be natural or synthetic. The poly-amino acids also may be modified, such that at least one atom has been added, removed, substituted, or combinations thereof (e.g., N-alkyl polyamino acid or N-acyl poly-amino acid). As used herein, poly-amino acids encompass both modified and unmodified poly-amino acids. For example, modified poly-amino acids include, but are not limited to, poly-amino acids of various molecular weights (MW), such as poly-L-α-glycine with a MW of about 100, about 200, about 300, about 500, about 1,000, about 1,500, about 6,000, about 25,200, about 63,000, about 83,000, or about 300,000 in Dalton (Da).

### BRIEF DESCRIPTION OF THE DRAWINGS

FIG. 1A-1B illustrates the Faster Hydration Benefit of the human study group using 3 different examples of the present disclosure and water (control) following thermal- and exercise-induced dehydration.

FIG. 2A-2B illustrates the Longer Lasting Hydration of the human study group using 3 different examples of the present disclosure and water (control) following thermal- and exercise-induced dehydration.

### DETAILED DESCRIPTION

The present disclosure is based, at least in part, on the findings that a formulation with at least one active ingredient, and in certain embodiments at least two active ingredients, can be used to rapidly improve the plasma D₂O percent after exercise and rehydration. A better sustained and replenished plasma D₂O percent—after rehydration—will provide possible thermoregulatory and performance advantages during subsequent exercise-heat stress.

The present disclosure advantageously provides compositions that are effective in improving the fast rehydration and retention of hydration, including long lasting hydration and/or long lasting rehydration.

In some embodiments, the composition is free of amino acids and/or substantially free of nutritive sweetener. In some embodiments, the composition comprises active ingredients. These active ingredients in the present oral composition include beverage compatible ingredients, such as at least one electrolyte and/or at least one sweetener and/or at least one functional ingredient and/or at least one additive.

### Electrolyte

The present composition may contain at least one electrolyte. Non limiting examples of electrolytes include sodium, potassium, calcium, magnesium, chloride, phosphate, bicarbonate, and combinations thereof. The electrolytes and ionic components for the present disclosure are usually, but not necessarily, obtainable from their corresponding water-soluble and non-toxic salts. Unless otherwise defined, the amount of electrolytes or ionic components in the composition is based on those present in the final ingestible form. The electrolyte concentration is of the ion only and not the salt.

The present composition preferably contains a total electrolyte concentration of at least about 200 mg/L, at least about 300 mg/L, at least about 400 mg/L, at least about 500 mg/L, at least about 600 mg/L, at least about 700 mg/L or at least about 800 mg/L. In a particular embodiment, the present composition contains an electrolyte concentration from about 400 mg/L to about 1,000 mg/L, from about 400 mg/L to about 900 mg/L, from about 400 mg/L to about 800 mg/L, from about 400 mg/L to about 700 mg/L, from about 400 mg/L to about 600 mg/L, from about 400 mg/L to about 500 mg/L, from about 500 mg/L to about 1,000 mg/L.

The potassium ion component can be provided by any salt including the chloride, carbonate, sulfate, acetate, bicarbonate, citrate, phosphate, hydrogen phosphate, tartrate, sorbate or a combination thereof. The potassium ions are preferably present in the composition of the present disclosure in an amount of at least 0.0025% to about 0.08% by weight, from about 0.0075% to about 0.06% or from about 0.0075% to about 0.015% by weight.

The present composition can contain from about 5 mg/L to about 1,000 mg/L potassium, more preferably from about 50 mg/L to about 300 mg/L, such as, for example, from about 100 mg/L to about 300 mg/L, from about 200 mg/L to about 300 mg/L, from about 50 mg/L to about 200 mg/L, from about 100 mg/L to about 200 mg/L or from about 100 mg/L to about 200 mg/L. In some embodiments, the oral composition has a concentration of at least one active ingredient comprising a mixture of potassium salts from about 15 mmol/L and about 35 mmol/L, or about between 17 mmol/L and about 30 mmol/L, or between about 23 mmol/L and about 29 mmol/L, or between about 25 mmol/L and about 28 mmol/L, or about 27.8 mmol/L.

The sodium ion component can be provided by any salt such as the chloride, carbonate, sulfate, acetate, bicarbonate, citrate, phosphate, hydrogen phosphate, tartrate, sorbate or a combination thereof. The sodium ions are preferably present in the present composition in an amount of at least about 0.005% to about 0.1% by weight, from about 0.0075% to about 0.075% or about 0.015% to about 0.05% by weight.

The present composition can contain from about 5 mg/L to about 1,000 mg/L sodium, more preferably from about 300 mg/L, to about 800 mg/L sodium, such as, for example, from about 300 mg/L to about 700 mg/L, from about 300 mg/L to about 600 mg/L, from about 300 mg/L to about 500 mg/L, from about 300 mg/L to about 400 mg/L, from about 400 mg/L to about 800 mg/L, from about 400 mg/L to about 700 mg/L, from about 400 mg/L to about 600 mg/L from about 400 mg/L to about 500 mg/l, from about 500 mg/L to about 800 mg/L, from about 500 mg/L to about 700 mg/L, from about 500 mg/L to about 600 mg/L, from about 600 mg/L to about 800 mg/L, from about 600 mg/L to about 700 mg/L and from about 700 mg/L to about 800 mg/L. In a particular embodiment, the present composition contains from about 600 mg/L to about 700 mg/L sodium. In some embodiments, the oral composition has a concentration of at least one active ingredient comprising a mixture of sodium salts from about 15 mmol/L and about 35 mmol/L, or about between 17 mmol/L and about 30 mmol/L, or between about 20 mmol/L and about 30 mmol/L, or between about 23 mmol/L and about 28 mmol/L, or between about 25 mmol/L, and about 26 mmol/L, or about 25.9 mmol/L.

The calcium ion component can be provided by any salt such as the chloride, carbonate, sulfate, acetate, bicarbonate, citrate, phosphate, hydrogen phosphate, tartrate, sorbate or a combination thereof. The calcium ions are preferably present in the present composition in an amount of at least about 0.0005% to about 0.010% by weight.

The present composition can contain from about 5 mg/L to about 1,000 mg/L calcium, more preferably from about 1 mg/L to about 50 mg/L, such as, for example, from about 5 mg/L to about 10 mg/L. The magnesium ion component can be provided by any salt such as the chloride, carbonate, sulfate, acetate, bicarbonate, citrate, phosphate, hydrogen phosphate, tartrate, sorbate or a combination thereof. The magnesium ions are preferably present in the present composition in an amount of at least about 0.0005% to about 0.010% by weight.

The present composition can contain from about 5 mg/L to about 1,000 mg/L magnesium, more preferably from about 1 mg/L to about 50 mg/L, such as, for example, from about 5 mg/L to about 20 mg/L.

The present composition can contain chloride ion from about 0.005% to about 0.20% by weight, from about 0.01% to about 0.15% or from about 0.02% to about 0.075%. The chloride ion component can be provided by a salt such as sodium chloride, potassium chloride or a combination thereof.

In a particular embodiment, a beverage of the present disclosure contains at least one electrolyte selected from the group consisting of sodium, potassium, magnesium, calcium and combinations thereof. In another particular embodiment, a beverage of the present disclosure contains at least one electrolyte selected from the group consisting of sodium, potassium, magnesium, calcium and combinations thereof, wherein the amount of each electrolyte is as provided above. In certain embodiments, the at least two active ingredients are a first active ingredient of sodium salts having a concentration between about 10 mmol/L and about 28 mmol/L, or about between 13 mmol/L. and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.

### Sweetener

The present composition may optionally include a sweetener. The sweetener can be an artificial or synthetic sweetener, a natural sweetener, a natural high potency sweetener. As used herein, the phrase “natural high potency sweetener” (NHPS) refers to any sweetener found naturally in nature and characteristically has a sweetness potency greater than sucrose, fructose, or glucose, yet has less calories. The natural high potency sweetener can be provided as a pure compound or, alternatively, as part of an extract. As used herein, the phrase “synthetic sweetener” refers to any composition which is not found naturally in nature and characteristically has a sweetness potency greater than sucrose, fructose, or glucose, yet has less calories. In certain embodiment the oral composition has no sweeteners. In certain embodiments, the oral composition is completely free and/or substantially free of nutritive sweeteners and/or non-nutritive sweeteners.

Non-limiting examples of NHPSs includes stevia and steviol glycosides, such as rebaudioside M, rebaudioside D, rebaudioside A, rebaudioside N, rebaudioside 0, rebaudioside E, steviolmonoside, steviolbioside, rubusoside, dulcoside B, dulcoside A, rebaudioside B, rebaudioside G, stevioside, rebaudioside C, rebaudioside F, rebaudioside I, rebaudioside H, rebaudioside L, rebaudioside K, rebaudioside J, rebaudioside M2, rebaudioside D2, rebaudioside S, rebaudioside T, rebaudioside U, rebaudioside V, rebaudioside W, rebaudioside Z1, rebaudioside Z2, rebaudioside IX, enzymatically glucosylated steviol glycosides and combinations thereof. Examples of high purity steviol glycosides and methods of making the same are provided in U.S. Pat. App. No. 2021/0107933, which is hereby incorporated in its entirety.

In certain embodiments, a steviol glycoside blend comprises at least about 5% steviol glycoside by weight, such as, for example, at least about 10%, at least about 20%, at least about 30%, at least about 40%, at least about 50%, at least about 60%, at least about 70%, at least about 80%, at least about 90%, at least about 95% or at least about 97%. In exemplary embodiments, the steviol glycoside blend comprises at least about 50% steviol glycoside by weight, such as, for example, from about 50% to about 90%, from about 50% to about 80%, from about 50% to about 70%, from about 50% to about 60%, from about 60% to about 90%, from about 60% to about 80%, from about 60% to about 70%, from about 70% to about 90%, from about 70% to about 80% and from about 80% to about 90%.

Another exemplary NHPS is Luo Han Guo and the related mogroside compounds, such as grosmogroside I, mogroside IA, mogroside IE, 11-oxomogroside IA, mogroside II mogroside II A, mogroside II B, mogroside II E, 7-oxomogroside II E, mogroside III, Mogroside HIE, 11-oxomogroside HIE, 11-deoxymogroside III, mogroside IV, Mogroside IVA 11-oxomogroside IV, 11-oxomogroside IVA, mogroside V, isomogroside V, 11-deoxymogroside V, 7-oxomogroside V, 11-oxomogroside V, isomogroside V, mogroside VI, mogrol, 11-oxomogrol, siamenoside I, isomers of siamenoside 1 (e.g. those disclosed in US Pat. App No 20170119032; incorporated by reference in its entirety), (3β, 9β, 10α, 11α, 24R)-3-[(4-O-β-D-glucospyranosyl-6-O-β-D-glucopyranosyl]-25-hydroxyl-9-methyl-19-norlanost-5-en-24-yl-[2-O-β-D-glucopyranosyl-6-O-β-D-glucopyranosyl]-β-D-glucopyranoside); (3β, 9β, 10α, 11α, 24R)-[(2-O-β-D-glucopyranosyl-6-O-β-D-glucopyranosyl-β-D-glucopyranosyl)oxy]-25-hydroxy-9-methyl-19-norlanost-5-en-24-yl-[2-O-β-D-glucopyranosyl-6-O-β-D-glucopyranosyl]-β-D-glucopyranoside); and (3p, 9β, 10α, 11α, 24R)-[(2-O-β-D-glucopyranosyl-6-O-β-D-glucopyranosyl-β-D-glucopyranosyl)oxy]-25-hydroxy-9-methyl-19-norlanost-5-en-24-yl-[2-O-β-D-glucopyranosyl-6-O-β-D-glucopyranosyl]-β-D-glucopyranoside).

In certain embodiments, a mogroside blend comprises at least about 5% of the mogroside by weight, such as, for example, at least about 10%, at least about 20%, at least about 30%, at least about 40%, at least about 50%, at least about 60%, at least about 70%, at least about 80%, at least about 90%, at least about 95% or at least about 97%.

Other exemplary NHPSs include monatin and its salts (monatin SS, RR, RS, SR), curculin, glycyrrhizic acid and its salts, thaumatin, monellin, mabinlin, brazzein, hemandulcin, phyllodulcin, glycyphyllin, phlordzin, trilobatin, baiyunoside, osladin, polypodoside A, pterocaryoside A, pterocaryoside B, mukurozioside, phlomisoside L periandrin L1 abrusoside A, and cyclocarioside 1.

In one embodiment, the sweetener is a carbohydrate sweetener. Suitable carbohydrate sweeteners include, but not limited to, the group consisting of sucrose, glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, fucose, rhamnose, arabinose, turanose, sialose and combinations thereof.

In certain embodiments, the present composition is free or substantially free from a carbohydrate sweetener.

Other suitable sweeteners include siamenoside, monatin and its salts (monatin SS, RR, RS, SR), curculin, mogrosides, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryosideB, mukurozioside, phlomisoside I, periandrin I, abrusoside A, steviolbioside and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs) and combinations thereof.

In one embodiment, the sweetener is a caloric sweetener or mixture of caloric sweeteners. In another embodiment, the caloric sweetener is selected from sucrose, fructose, glucose, high fructose com/starch syrup, a beet sugar, a cane sugar, and combinations thereof.

In certain embodiments, the present composition is free or substantially free from a caloric sweetener.

In another embodiment, the sweetener is a rare sugar selected from allulose, gulose, kojibiose, sorbose, lyxose, ribulose, xylose, xylulose, D-allose, L-ribose, D-tagatose, L-glucose, L-fucose, L-arabinose, turanose and combinations thereof.

The amount of sweetener in the present composition depends on the identity of the sweetener and the desired level of sweetness. In preferred embodiments, the sweetener is present in a sweetening amount, i.e. a concentration that is detectably sweet.

As would be understood by a person of skill in the art, high potency sweeteners are more potent and therefore lower concentrations are required to achieve a particular sucrose equivalence (SE). The sweetness of a non-sucrose sweetener can be measured against a sucrose reference by determining the non-sucrose sweetener's sucrose equivalence (SE). Typically, taste panelists are trained to detect sweetness of reference sucrose solutions containing between 1-15% sucrose (w/v). Other non-sucrose sweeteners are then tasted at a series of dilutions to determine the concentration of the non-sucrose sweetener that is as sweet as a given percent sucrose reference. For example, if a 1% solution of a non-sucrose sweetener is as sweet as a 10% sucrose solution, then the sweetener is said to be 10 times as potent as sucrose, and has 10% sucrose equivalence.

In one embodiment, the sweetener or sweeteners provides the present composition with a sucrose equivalence of about 1% (w/v), such as, for example, about 2%, about 3%, about 4%, about 5%, about 6%, about 7%, about 8%, about 9%, about 10%, about 11%, about 12%, about 13%, about 14% or any range between these values.

In another embodiment, the present composition has a SE from about 2% to about 14%, such as, for example, from about 2% to about 10%, from about 2% to about 5%, from about 5% to about 15%, from about 5% to about 10% or from about 10% to about 15%.

The amount of sucrose, and thus another measure of sweetness, in a reference solution may be described in degrees Brix (° Bx). One degree Brix is 1 gram of sucrose in 100 grams of solution and represents the strength of the solution as percentage by weight (% w/w) (strictly speaking, by mass). In embodiments where the present composition is sweetened with sucrose, the beverage can be about 1 degree Brix, about 2 degrees Brix, about 3 degrees Brix, about 4 degrees Brix, about 5 degrees Brix, about 6 degrees Brix, about 7 degrees Brix, about 8 degrees Brx, about 9 degrees Brix, about degrees Brix, about 11 degrees Brix, about 12 degrees Brix, about 13 degrees Brix, about 14 degrees Brix or any range between these values.

### Functional Components/Ingredients

The present composition may optionally include a functional ingredient. Exemplary functional ingredients include, but are not limited to, saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

In certain embodiments, the functional ingredient is at least one saponin. As used herein, the at least one saponin may comprise a single saponin or a plurality of saponins as a functional ingredient for the composition provided herein. Saponins are glycosidic natural plant products comprising an aglycone ring structure and one or more sugar moieties. Non-limiting examples of specific saponins for use in particular embodiments of the disclosure include group A acetyl saponin, group B acetyl saponin, and group E acetyl saponin. Several common sources of saponins include soybeans, which have approximately 5% saponin content by dry weight, soapwort plants Saponaria), the root of which was used historically as soap, as well as alfalfa, aloe, asparagus, grapes, chickpeas, yucca, and various other beans and weeds. Saponins may be obtained from these sources by using extraction techniques well known to those of ordinary skill in the art A description of conventional extraction techniques can be found in U.S. Pat. Appl. No. 2005/0123662.

In certain embodiments, the functional ingredient is at least one antioxidant. As used herein, “antioxidant” refers to any substance which inhibits, suppresses, or reduces oxidative damage to cells and biomolecules.

Examples of suitable antioxidants for embodiments of this disclosure include, but are not limited to, vitamins, vitamin cofactors, minerals, hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, and combinations thereof. In some embodiments, the antioxidant is vitamin A, vitamin C, vitamin E, ubiquinone, mineral selenium, manganese, melatonin, oc-carotene, b-carotene, lycopene, lutein, zeanthin, crypoxanthin, reservatol, eugenol, quercetin, catechin, gossypol, hesperetin, curcumin, ferulic acid, thymol, hydroxytyrosol, turmeric, thyme, olive oil, lipoic acid, glutathinone, gutamine, oxalic acid, tocopherol-derived compounds, butylated hydroxyanisole (BHA), butylated hydroxytoluene (BHT), ethylenediaminetetraacetic acid (EDTA), tert-butylhydroquinone, acetic acid, pectin, tocotrienol, tocopherol, coenzyme Q10, zeaxanthin, astaxanthin, canthaxantin, saponins, limonoids, kaempfedrol, myricetin, isorhamnetin, proanthocyanidins, quercetin, rutin, luteolin, apigenin, tangeritin, hesperetin, naringenin, erodictyol, flavan-3-ols (e.g., anthocyanidins), gallocatechins, epicatechin and its gallate forms, epigallocatechin and its gallate forms (ECGC) theaflavin and its gallate forms, thearubigins, isoflavone, phytoestrogens, genistein, daidzein, glycitein, anythocyanins, cyaniding, delphinidin, malvidin, pelargonidin, peonidin, petunidin, ellagic acid, gallic acid, salicylic acid, rosmarinic acid, cinnamic acid and its derivatives (e.g., ferulic acid), chlorogenic acid, chicoric acid, gallotannins, ellagitannins, anthoxanthins, betacyanins and other plant pigments, silymarin, citric acid, lignan, antinutrients, bilirubin, uric acid, R-oc-lipoic acid, N-acetylcysteine, emblicanin, apple extract, apple skin extract (applephenon), rooibos extract red, rooibos extract, green, hawthorn berry extract, red raspberry extract, green coffee antioxidant (GCA), aronia extract 20/o, grape seed extract (VinOseed), cocoa extract, hops extract, mangosteen extract, mangosteen hull extract, cranberry extract, pomegranate extract, pomegranate hull extract, pomegranate seed extract, hawthorn berry extract, pomella pomegranate extract, cinnamon bark extract, grape skin extract, bilberry extract, pine bark extract, pycnogenol, elderberry extract, mulberry root extract, wolfberry (gogi) extract, blackberry extract, blueberry extract, blueberry leaf extract, raspberry extract, turmeric extract, citrus bioflavonoids, black currant, ginger, acai powder, green coffee bean extract, green tea extract, and phytic acid, or combinations thereof. In alternate embodiments, the antioxidant is a synthetic antioxidant such as butylated hydroxytolune or butylated hydroxyanisole, for example. Other sources of suitable antioxidants for embodiments of this disclosure include, but are not limited to, fruits, vegetables, tea, cocoa, chocolate, spices, herbs, rice, organ meats from livestock, yeast, whole grains, or cereal grains.

Particular antioxidants belong to the class of phytonutrients called polyphenols (also known as “polyphenolics”), which are a group of chemical substances found in plants, characterized by the presence of more than one phenol group per molecule. Suitable polyphenols for embodiments of this disclosure include catechins, proanthocyanidins, procyanidins, anthocyanins, quercerin, rutin, reservatrol, isotlavones, curcumin, punicalagin, ellagitannin, hesperidin, naringin, citrus flavonoids, chlorogenic acid, other similar materials, and combinations thereof.

In one embodiment, the antioxidant is a catechin such as, for example, epigallocatechin gallate (EGCG). In another embodiment, the antioxidant is chosen from proanthocyanidins, procyanidins or combinations thereof. In particular embodiments, the antioxidant is an anthocyanin. In still other embodiments, the antioxidant is chosen from quercetin, rutin or combinations thereof. In one embodiment, the antioxidant is reservatrol. In another embodiment, the antioxidant is an isoflavone. In still another embodiment, the antioxidant is curcumin. In a yet further embodiment, the antioxidant is chosen from punicalagin, ellagitannin or combinations thereof. In a still further embodiment, the antioxidant is chlorogenic acid.

In certain embodiments, the functional ingredient is at least one dietary fiber. Numerous polymeric carbohydrates having significantly different structures in both composition and linkages fall within the definition of dietary fiber. Such compounds are well known to those skilled in the art, non-limiting examples of which include non-starch polysaccharides, lignin, cellulose, methylcellulose, the hemicelluloses, b-glucans, pectins, gums, mucilage, waxes, inulins, oligosaccharides, fructooligosaccharides, cyclodextrins, chitins, and combinations thereof. Although dietary fiber generally is derived from plant sources, indigestible animal products such as chitins are also classified as dietary fiber. Chitin is a polysaccharide composed of units of acetylglucosamine joined by b(1-4) linkages, similar to the linkages of cellulose.

In certain embodiments, the functional ingredient is at least one fatty acid. As used herein, “fatty acid” refers to any straight chain monocarboxylic acid and includes saturated fatty acids, unsaturated fatty acids, long chain fatty acids, medium chain fatty acids, short chain fatty acids, fatty acid precursors (including omega-9 fatty acid precursors), and esterified fatty acids. As used herein, “long chain polyunsaturated fatty acid” refers to any polyunsaturated carboxylic acid or organic acid with a long aliphatic tail. As used herein, “omega-3 fatty acid” refers to any polyunsaturated fatty acid having a first double bond as the third carbon-carbon bond from the terminal methyl end of its carbon chain. In particular embodiments, the omega-3 fatty acid may comprise a long chain omega-3 fatty acid. As used herein, “omega-6 fatty acid” any polyunsaturated fatty acid having a first double bond as the sixth carbon-carbon bond from the terminal methyl end of its carbon chain.

Suitable omega-3 fatty acids for use in embodiments of the present disclosure can be derived from algae, fish, animals, plants, or combinations thereof, for example. Examples of suitable omega-3 fatty acids include, but are not limited to, linolenic acid, alpha-linolenic acid, eicosapentaenoic acid, docosahexaenoic acid, stearidonic acid, eicosatetraenoic acid and combinations thereof. In some embodiments, suitable omega-3 fatty acids can be provided in fish oils, (e.g., menhaden oil, tuna oil, salmon oil, bonito oil, and cod oil), microalgae omega-3 oils or combinations thereof. In particular embodiments, suitable omega-3 fatty acids may be derived from commercially available omega-3 fatty acid oils such as Microalgae DHA oil (from Martek, Columbia, MD), OmegaPure (from Omega Protein, Houston, TX), Marinol C-38 (from Lipid Nutrition, Channahon, IL), Bonito oil and MEG-3 (from Ocean Nutrition, Dartmouth, NS), Evogel (from Symrise, Holzminden, Germany), Marine Oil, from tuna or salmon (from Arista Wilton, CT), OmegaSource 2000, Marine Oil, from menhaden and Marine Oil, from cod (from OmegaSource, RTP, NC). Suitable omega-6 fatty acids include, but are not limited to, linoleic acid, gamma-linolenic acid, dihommo-gamma-linolenic acid, arachidonic acid, eicosadienoic acid, docosadienoic acid, adrenic acid, docosapentaenoic acid and combinations thereof.

Suitable esterified fatty acids for embodiments of the present disclosure include, but are not limited to, monoacylgycerols containing omega-3 and/or omega-6 fatty acids, diacylgycerols containing omega-3 and/or omega-6 fatty acids, or triacylgycerols containing omega-3 and/or omega-6 fatty acids and combinations thereof.

In certain embodiments, the functional ingredient is at least one vitamin. Suitable vitamins include, vitamin A, vitamin D, vitamin E, vitamin K, vitamin B1, vitamin B2, vitamin B3, vitamin B5, vitamin B6, vitamin B7, vitamin B9, vitamin B 12, and vitamin C.

Various other compounds have been classified as vitamins by some authorities. These compounds may be tenned pseudo-vitamins and include, but are not limited to, compounds such as ubiquinone (coenzyme Q10), pangamic acid, dimethylglycine, taestrile, amygdaline, flavanoids, para-aminobenzoic acid, adenine, adenylic acid, and s-methylmethionine. As used herein, the term vitamin includes pseudo-vitamins. In some embodiments, the vitamin is a fat-soluble vitamin chosen from vitamin A, D, E, K and combinations thereof. In other embodiments, the vitamin is a water-soluble vitamin chosen from vitamin B1, vitamin B2, vitamin B3, vitamin B6, vitamin B12, folic acid, biotin, pantothenic acid, vitamin C and combinations thereof.

In certain embodiments, the functional ingredient is glucosamine, optionally further comprising chondroitin sulfate.

In certain embodiments, the functional ingredient is at least one mineral. Minerals, in accordance with the teachings of this disclosure, comprise inorganic chemical elements required by living organisms. Minerals are comprised of a broad range of compositions (e.g., elements, simple salts, and complex silicates) and also vary broadly in crystalline structure. They may naturally occur in foods and beverages, may be added as a supplement, or may be consumed or administered separately from foods or beverages.

Minerals may be categorized as either bulk minerals, which are required in relatively large amounts, or trace minerals, which are required in relatively small amounts. Bulk minerals generally are required in amounts greater than or equal to about 100 mg per day and trace minerals are those that are required in amounts less than about 100 mg per day. In one embodiment, the mineral is chosen from bulk minerals, trace minerals or combinations thereof. Non-limiting examples of bulk minerals include calcium, chlorine, magnesium, phosphorous, potassium, sodium, and sulfur. Non-limiting examples of trace minerals include chromium, cobalt, copper, fluorine, iron, manganese, molybdenum, selenium, zinc, and iodine. Although iodine generally is classified as a trace mineral, it is required in larger quantities than other trace minerals and often is categorized as a bulk mineral.

In a particular embodiment, the mineral is a trace mineral, believed to be necessary for human nutrition, non-limiting examples of which include bismuth, boron, lithium, nickel, rubidium, silicon, strontium, tellurium, tin, titanium, tungsten, and vanadium.

The minerals embodied herein may be in any form known to those of ordinary skill in the art. For example, in one embodiment, the minerals may be in their ionic form, having either a positive or negative charge. In another embodiment, the minerals may be in their molecular form. For example, sulfur and phosphorous often are found naturally as sulfates, sulfides, and phosphates.

In certain embodiments, the functional ingredient is at least one preservative. In particular embodiments, the preservative is chosen from antimicrobials, antioxidants, antienzymatics or combinations thereof. Non-limiting examples of antimicrobials include sulfites, propionates, benzoates, sorbates, nitrates, nitrites, bacteriocins, salts, sugars, acetic acid, dimethyl dicarbonate (DMDC), ethanol, and ozone. In one embodiment, the preservative is a sulfite. Sulfites include, but are not limited to, sulfur dioxide, sodium bisulfite, and potassium hydrogen sulfite. In another embodiment, the preservative is a propionate. Propionates include, but are not limited to, propionic acid, calcium propionate, and sodium propionate. In yet another embodiment, the preservative is a benzoate. Benzoates include, but are not limited to, sodium benzoate and benzoic acid. In still another embodiment, the preservative is a sorbate. Sorbates include, but are not limited to, potassium sorbate, sodium sorbate, calcium sorbate, and sorbic acid. In a still further embodiment, the preservative is a nitrate and/or a nitrite. Nitrates and nitrites include, but are not limited to, sodium nitrate and sodium nitrite. In another embodiment, the at least one preservative is a bacteriocin, such as, for example, nisin. In still another embodiment, the preservative is ethanol. In yet another embodiment, the preservative is ozone. Non-limiting examples of anti-enzymatis suitable for use as preservatives in particular embodiments of the disclosure include ascorbic acid, citric acid, and metal chelating agents such as ethylenediaminetetraacetic acid (EDTA).

In certain embodiments, the functional ingredient is an additional hydration agent to the amino acid-free formulation. The additional hydration agent can be a synergist when combined with the amino acid-free formulation further enhances the hydration or rehydration effect of the composition. In one particular embodiment, the additional hydration agent is a carbohydrate to supplement energy stores burned by muscles. Suitable carbohydrates for use in particular embodiments of this disclosure are described in U.S. Pat. Nos. 4,312,856, 4,853,237, 5,681,569, and 6,989,171. Non-limiting examples of suitable carbohydrates include monosaccharides, disaccharides, oligosaccharides, complex polysaccharides or combinations thereof. Non-limiting examples of suitable types of monosaccharides for use in particular embodiments include trioses, tetroses, pentoses, hexoses, heptoses, octoses, and nonoses. Non-limiting examples of specific types of suitable monosaccharides include glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, and sialose. Non-limiting examples of suitable disaccharides include sucrose, lactose, and maltose. Non-limiting examples of suitable oligosaccharides include saccharose, maltotriose, and maltodextrin. In other particular embodiments, the carbohydrates are provided by a corn syrup, a beet sugar, a cane sugar, a juice, or a tea.

In another particular embodiment, the additional hydration agent is a flavanol that provides cellular rehydration. Flavanols are a class of natural substances present in plants, and generally comprise a 2-phenylbenzopyrone molecular skeleton attached to one or more chemical moieties. Non limiting examples of suitable flavanols for use in particular embodiments of this disclosure include catechin, epicatechin, gallocatechin, epigallocatechin, epicatechin gallate, epigallocatechin 3-gallate, theaflavin, theaflavin 3-gallate, theaflavin 3′-gallate, theaflavin 3,3′ gallate, thearubigin or combinations thereof. Several common sources of flavanols include tea plants, fruits, vegetables, and flowers. In preferred embodiments, the flavanol is extracted from green tea.

In a particular embodiment, the additional hydration agent is a glycerol solution to enhance exercise endurance. The ingestion of a glycerol containing solution has been shown to provide beneficial physiological effects, such as expanded blood volume, lower heart rate, and lower rectal temperature.

In certain embodiments, the functional ingredient is chosen from at least one probiotic, prebiotic and combination thereof. The probiotic is a beneficial microorganism that affects the human body's naturally-occurring gastrointestinal microflora. Examples of probiotics include, but are not limited to, bacteria of the genus Lactobacilli, Bifidobacteria, Streptococci, or combinations thereof, that confer beneficial effects to humans. In particular embodiments of the disclosure, the at least one probiotic is chosen from the genus Lactobacilli. According to other particular embodiments of this disclosure, the probiotic is chosen from the genus Bifidobacteria. In a particular embodiment, the probiotic is chosen from the genus Streptococcus.

Probiotics that may be used in accordance with this disclosure are well-known to those of skill in the art. Non-limiting examples of foodstuffs comprising probiotics include yogurt, sauerkraut, kefir, kimchi, fermented vegetables, and other foodstuffs containing a microbial element that beneficially affects the host animal by improving the intestinal microbalance.

Prebiotics, in accordance with the embodiments of this disclosure, include, without limitation, mucopolysaccharides, oligosaccharides, polysaccharides, vitamins, nutrient precursors, proteins and combinations thereof. According to a particular embodiment of this disclosure, the prebiotic is chosen from dietary fibers, including, without limitation, polysaccharides and oligosaccharides. Non-limiting examples of oligosaccharides that are categorized as prebiotics in accordance with particular embodiments of this disclosure include fructooligosaccharides, inulins, isomalto-oligosaccharides, lactilol, lactosucrose, lactulose, pyrodextrins, soy oligosaccharides, transgalacto-oligosaccharides, and xylo-oligosaccharides.

Prebiotics are found naturally in a variety of foods including, without limitation, bananas, berries, asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans).

In certain embodiments, the functional ingredient is at least one weight management agent. As used herein, “a weight management agent” includes an appetite suppressant and/or a thermogenesis agent. As used herein, the phrases “appetite suppressant”, “appetite satiation compositions”, “satiety agents”, and “satiety ingredients” are synonymous. The phrase “appetite suppressant” describes macronutrients, herbal extracts, exogenous hormones, anorectics, anorexigenics, pharmaceutical drugs, and combinations thereof, that when delivered in an effective amount, suppress, inhibit, reduce, or otherwise curtail a person's appetite. The phrase “thermogenesis agent” describes macronutrients, herbal extracts, exogenous hormones, anorectics, anorexigenics, pharmaceutical drugs, and combinations thereof, that when delivered in an effective amount, activate or otherwise enhance a person's thermogenesis or metabolism.

Suitable weight management agents include macronutrients selected from the group consisting of proteins, carbohydrates, dietary fats, and combinations thereof. Consumption of proteins, carbohydrates, and dietary fats stimulates the release of peptides with appetite suppressing effects. For example, consumption of proteins and dietary fats stimulates the release of the gut hormone cholecytokinin (CCK), while consumption of carbohydrates and dietary fats stimulates release of Glucagon-like peptide 1 (GLP-1).

Suitable macronutrient weight management agents also include carbohydrates. Carbohydrates generally comprise sugars, starches, cellulose and gums that the body converts into glucose for energy. Carbohydrates often are classified into two categories, digestible carbohydrates (e.g., monosaccharides, disaccharides, and starch) and non-digestible carbohydrates (e.g., dietary fiber). Studies have shown that non-digestible carbohydrates and complex polymeric carbohydrates having reduced absorption and digestibility in the small intestine stimulate physiologic responses that inhibit food intake. Accordingly, the carbohydrates embodied herein desirably comprise non-digestible carbohydrates or carbohydrates with reduced digestibility. Non-limiting examples of such carbohydrates include polydextrose; inulin; monosaccharide-derived polyols such as erythritol, mannitol, xylitol, and sorbitol; disaccharide-derived alcohols such as isomalt, lactitol, and maltitol; and hydrogenated starch hydrolysates. Carbohydrates are described in more detail herein below.

In another particular embodiment, the weight management agent is a dietary fat. Dietary fats are lipids comprising combinations of saturated and unsaturated fatty acids. Polyunsaturated fatty acids have been shown to have a greater satiating power than mono-unsaturated fatty acids. Accordingly, the dietary fats embodied herein desirably comprise poly-unsaturated fatty acids, non-limiting examples of which include triacylglycerols. In another particular embodiment, the weight management agent is an herbal extract. Extracts from numerous types of plants have been identified as possessing appetite suppressant properties. Non-limiting examples of plants whose extracts have appetite suppressant properties include plants of the genus Hoodia, Trichocaulon, Caralluma, Stapelia, Orbea, Asclepias, and Camelia. Other embodiments include extracts derived from Gymnema Sylvestre, Kola Nut, Citrus Auran tium, Yerba Mate, Griffonia Simplicifolia, Guarana, myrrh, guggul Lipid, and black current seed oil.

The herbal extracts may be prepared from any type of plant material or plant biomass. Non-limiting examples of plant material and biomass include the stems, roots, leaves, dried powder obtained from the plant material, and sap or dried sap. The herbal extracts generally are prepared by extracting sap from the plant and then spray-drying the sap. Alternatively, solvent extraction procedures may be employed. Following the initial extraction, it may be desirable to further fractionate the initial extract (e.g., by column chromatography) in order to obtain an herbal extract with enhanced activity. Such techniques are well known to those of ordinary skill in the art.

In one embodiment, the herbal extract is derived from a plant of the genus Hoodia. A sterol glycoside of Hoodia, known as P57, is believed to be responsible for the appetite-suppressant effect of the Hoodia species. In another embodiment, the herbal extract is derived from a plant of the genus Caralluma, non-limiting examples of which include caratuberside A, caratuberside B, bouceroside I, bouceroside II, bouceroside III, bouceroside IV, bouceroside V, bouceroside VI, bouceroside VII, bouceroside VIII, bouceroside IX, and bouceroside X. In another embodiment, the at least one herbal extract is derived from a plant of the genus Trichocaulon. Trichocaulon plants are succulents that generally are native to southern Africa, similar to Hoodia, and include the species T piliferum and T officinale. In another embodiment, the herbal extract is derived from a plant of the genus Stapelia or Orbea. Not wishing to be bound by any theory, it is believed that the compounds exhibiting appetite suppressant activity are saponins, such as pregnane glycosides, which include stavarosides A, B, C, D, E, F, G, H, I, J, and K. In another embodiment, the herbal extract is derived from a plant of the genus Asclepias. Not wishing to be bound by any theory, it is believed that the extracts comprise steroidal compounds, such as pregnane glycosides and pregnane aglycone, having appetite suppressant effects. In another particular embodiment, the weight management agent is an exogenous hormone having a weight management effect. Non-limiting examples of such hormones include CCK, peptide YY, ghrelin, bombesin and gastrin-releasing peptide (GRP), enterostatin, apolipoprotein A-IV, GLP-1, amylin, somastatin, and leptin.

In another embodiment, the weight management agent is a pharmaceutical drug. Non limiting examples include phentenime, diethylpropion, phendimetrazine, sibutramine, rimonabant, oxyntomodulin, floxetine hydrochloride, ephedrine, phenethylamine, or other stimulants.

In certain embodiments, the functional ingredient is at least one osteoporosis management agent. In certain embodiments, the osteoporosis management agent is at least one calcium source. According to a particular embodiment, the calcium source is any compound containing calcium, including salt complexes, solubilized species, and other forms of calcium Non-limiting examples of calcium sources include calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium citrate malate, calcium gluconate, calcium tartrate, calcium lactate, solubilized species thereof, and combinations thereof.

According to a particular embodiment, the osteoporosis management agent is a magnesium source. The magnesium source is any compound containing magnesium, including salt complexes, solubilized species, and other forms of magnesium. Non-limiting examples of magnesium sources include magnesium chloride, magnesium citrate, magnesium gluceptate, magnesium gluconate, magnesium lactate, magnesium hydroxide, magnesium picolate, magnesium sulfate, solubilized species thereof, and mixtures thereof.

In other embodiments, the osteoporosis agent is chosen from vitamins D, C, K, their precursors and/or beta-carotene and combinations thereof.

Numerous plants and plant extracts also have been identified as being effective in the prevention and treatment of osteoporosis. Non-limiting examples of suitable plants and plant extracts as osteoporosis management agents include species of the genus Taraxacum and Amelanchier, as disclosed in U.S. Patent Publication No. 2005/0106215, and species of the genus Lindera, Artemisia, Acorus, Carthamus, Carum, Cnidium, Curcuma, Cyperus, Juniperus, Prunus, Iris, Cichorium, Dodonaea, Epimedium, Erigonoum, rioya, Mentha, Ocimum, thymus, Tanacetum, Plantago, Spearmint, Bixa, Vitis, Rosemarinus, Rhus, and Anethum, as disclosed in U.S. Pat. App. No. 2005/0079232.

In certain embodiments, the functional ingredient is at least one phytoestrogen. Phytoestrogens are compounds found in plants which can typically be delivered into human bodies by ingestion of the plants or the plant parts having the phytoestrogens. As used herein, “phytoestrogen” refers to any substance which, when introduced into a body causes an estrogen like effect of any degree. For example, a phytoestrogen may bind to estrogen receptors within the body and have a small estrogen-like effect.

Examples of suitable phytoestrogens for embodiments of this disclosure include, but are not limited to, isoflavones, stilbenes, lignans, resorcyclic acid lactones, coumestans, coumestrol, equol, and combinations thereof. Sources of suitable phytoestrogens include, but are not limited to, whole grains, cereals, fibers, fruits, vegetables, black cohosh, agave root, black currant, black haw, chasteberries, cramp bark, dong quai root, devil's club root, false unicorn root, ginseng root, groundsel herb, licorice, liferoot herb, motherwort herb, peony root, raspberry leaves, rose family plants, sage leaves, sarsaparilla root, saw palmetto berried, wild yam root, varrow blossoms, legumes, soybeans, soy products (e.g., miso, soy flour, soymilk, soy nuts, soy protein isolate, tempen, or tofu) chick peas, nuts, lentils, seeds, clover, red clover, dandelion leaves, dandelion roots, fenugreek seeds, green tea, hops, red wine, flaxseed, garlic, onions, linseed, borage, butterfly weed, caraway, chaste tree, vitex, dates, dill, fennel seed, gotu kola, milk thistle, pennyroyal, pomegranates, southernwood, soya flour, tansy, and root of the kudzu vine (pueraria root) and the like, and combinations thereof.

Isoflavones belong to the group of phytonutrients called polyphenols. In general, polyphenols (also known as “polyphenolics”), are a group of chemical substances found in plants, characterized by the presence of more than one phenol group per molecule.

Suitable phytoestrogen isoflavones in accordance with embodiments of this disclosure include genistein, daidzein, glycitein, biochanin A, formononetin, their respective naturally occurring glycosides and glycoside conjugates, matairesinol, secoisolariciresinol, enter olactone, enterodiol, textured vegetable protein, and combinations thereof. Suitable sources of isoflavones for embodiments of this disclosure include, but are not limited to, soybeans, soy products, legumes, alfalfa sprouts, chickpeas, peanuts, and red clover.

In certain embodiments, the functional ingredient is at least one long chain primary aliphatic saturated alcohol Long-chain primary aliphatic saturated alcohols are a diverse group of organic compounds. The term alcohol refers to the fact these compounds feature a hydroxyl group (—OH) bound to a carbon atom. Non-limiting examples of particular long-chain primary aliphatic saturated alcohols for use in particular embodiments of the disclosure include the 8 carbon atom 1-octanol, the 9 carbon 1-nonanol, the 10 carbon atom 1-decanol, the 12 carbon atom 1-dodecanol, the 14 carbon atom 1-tetradecanol, the 16 carbon atom 1-hexadecanol, the 18 carbon atom 1-octadecanol, the 20 carbon atom 1-eicosanol, the 22 carbon 1-docosanol, the 24 carbon 1-tetracosanol, the 26 carbon 1-hexacosanol, the 27 carbon 1-heptacosanol, the 28 carbon 1-octanosol, the 29 carbon 1-nonacosanol, the 30 carbon 1-triacontanol, the 32 carbon 1-dotriacontanol, and the 34 carbon 1-tetracontanol.

In one embodiment, the long-chain primary aliphatic saturated alcohol is a policosanol. Policosanol is the term for a mixture of long-chain primary aliphatic saturated alcohols composed primarily of 28 carbon 1-octanosol and 30 carbon 1-triacontanol, as well as other alcohols in lower concentrations such as 22 carbon 1-docosanol, 24 carbon 1-tetracosanol, 26 carbon 1-hexacosanol, 27 carbon 1-heptacosanol, 29 carbon 1-nonacosanol, 32 carbon 1-dotriacontanol, and 34 carbon 1-tetracontanol.

In certain embodiments, the functional ingredient is at least one phytosterol, phytostanol or combination thereof. As used herein, the phrases “stanol”, “plant stanol” and “phytostanol” are synonymous. Plant sterols and stands are present naturally in small quantities in many fruits, vegetables, nuts, seeds, cereals, legumes, vegetable oils, bark of the trees and other plant sources. Sterols are a subgroup of steroids with a hydroxyl group at C-3. Generally, phytosterols have a double bond within the steroid nucleus, like cholesterol; however, phytosterols also may comprise a substituted side chain (R) at C-24, such as an ethyl or methyl group, or an additional double bond. The structures of phytosterols are well known to those of skill in the art.

At least 44 naturally-occurring phytosterols have been discovered, and generally are derived from plants, such as corn, soy, wheat, and wood oils; however, they also may be produced synthetically to form compositions identical to those in nature or having properties similar to those of naturally-occurring phytosterols. Non-limiting suitable phytosterols include, but are not limited to, 4-desmethylsterols (e.g., b-sitosterol, campesterol, stigmasterol, brassicasterol, 22-dehydrobrassicasterol, and A5-avenasterol), 4-monomethyl sterols, and 4,4-dimethyl sterols (triterpene alcohols)(e.g., cycloartenol, 24-methylenecycloartanol, and cyclobranol).

As used herein, the phrases “stanol”, “plant stanol” and “phytostanol” are synonymous. Phytostanols are saturated sterol alcohols present in only trace amounts in nature and also may be synthetically produced, such as by hydrogenation of phytosterols. Suitable phytostanols include, but are not limited to, b-sitostanol, campestanol, cycloartanol, and saturated forms of other triterpene alcohols.

Both phytosterols and phytostanols, as used herein, include the various isomers such as the a and b isomers. The phytosterols and phytostanols of the present disclosure also may be in their ester form. Suitable methods for deriving the esters of phytosterols and phytostanols are well known to those of ordinary skill in the art, and are disclosed in U.S. Pat. Nos. 6,589,588, 6,635,774, 6,800,317, and U.S. Pat. App. No. 2003/0045473. Non limiting examples of suitable phytosterol and phytostanol esters include sitosterol acetate, sitosterol oleate, stigmasterol oleate, and their corresponding phytostanol esters. The phytosterols and phytostanols of the present disclosure also may include their derivatives.

Exemplary additives include, but not limited to, carbohydrates, polyols, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, plant extracts, flavonoids, alcohols, polymers and combinations thereof.

In one embodiment, the composition further comprises one or more polyols. The term “polyol”, as used herein, refers to a molecule that contains more than one hydroxyl group. A polyol may be a diol, triol, or a tetrad which contains 2, 3, and 4 hydroxyl groups respectively.

A polyol also may contain more than 4 hydroxyl groups, such as a pentad, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Non-limiting examples of polyols in some embodiments include maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, and sugar alcohols or any other carbohydrates capable of being reduced which do not adversely affect taste.

Suitable sugar acid additives include, but are not limited to, aldonic, uronic, aldaric, alginic, gluconic, glucuronic, glucaric, galactaric, galacturonic, and salts thereof (e.g., sodium, potassium, calcium, magnesium salts or other physiologically acceptable salts), and combinations thereof.

Suitable nucleotide additives include, but are not limited to, inosine monophosphate (IMP), guanosine monophosphate (GMP), adenosine monophosphate (AMP), cytosine monophosphate (CMP), uracil monophosphate (UMP), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g., guanine, cytosine, adenine, thymine, uracil).

Suitable organic acid additives include any compound which comprises a —COOH moiety, such as, for example, C2-C30 carboxylic acids, substituted hydroxyl C2-C30 carboxylic acids, butyric acid (ethyl esters), substituted butyric acid (ethyl esters), benzoic acid, substituted benzoic acids (e.g., 2,4-dihydroxybenzoic acid), substituted cinnamic acids, hydroxyacids, substituted hydroxybenzoic acids, anisic acid substituted cyclohexyl carboxylic acids, tannic acid, aconitic acid, lactic acid, tartaric acid, citric acid, isocitric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, catTeic acid, bile acids, acetic acid, ascorbic acid, alginic acid, erythorbic acid, polyglutamic acid, glucono delta lactone, and their alkali or alkaline earth metal salt derivatives thereof. In addition, the organic acid additives also may be in either the D- or L-configuration. Suitable organic acid additive salts include, but are not limited to, sodium, calcium, potassium, and magnesium salts of all organic acids, such as salts of citric acid, malic acid, tartaric acid, fumaric acid, lactic acid (e.g., sodium lactate), alginic acid (e g, sodium alginate), ascorbic acid (e.g, sodium ascorbate), benzoic acid (e.g, sodium benzoate or potassium benzoate), sorbic acid and adipic acid. The examples of the organic acid additives described optionally may be substituted with at least one group chosen from hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfnyl, sulfamyl, carboxalkoxy, carboxamido, phosphonyl, phosphinyl, phosphoryl, phosphino, thioester, thioether, anhydride, oximino, hydrazino, carbamyl, phosphor or phosphonato. In particular embodiments, the organic acid additive is present in the sweetener composition in an amount effective to provide a concentration from about 10 ppm to about 5,000 ppm when present in a consumable, such as, for example, a beverage.

Suitable inorganic acid additives include, but are not limited to, phosphoric acid, phosphorous acid, polyphosphoric acid, hydrochloric acid, sulfuric acid, carbonic acid, sodium dihydrogen phosphate, and alkali or alkaline earth metal salts thereof (e.g. inositol hexaphosphate Mg/Ca).

Suitable bitter compound additives include, but are not limited to, caffeine, quinine, urea, bitter orange oil, naringin, quassia, and salts thereof.

Suitable flavorants and flavoring ingredient additives include, but are not limited to, vanillin, vanilla extract, mango extract, cinnamon, citrus, coconut, ginger, viridiflorol, almond, menthol (including menthol without mint), grape skin extract, and grape seed extract. “Flavorant” and “flavoring ingredient” are synonymous and can include natural or synthetic substances or combinations thereof. Flavorants also include any other substance which imparts flavor and may include natural or non-natural (synthetic) substances which are safe for human or animals when used in a generally accepted range. Non-limiting examples of proprietary flavorants include DOHLERM Natural Flavoring Sweetness Enhancer K14323 (DOHLER™, Darmstadt, Germany), Symrise™ Natural Flavor Mask for Sweeteners 161453 and 164126 (SYMRISE™, Holzminden, Germany), Natural Advantage™ Bitterness Blockers 1, 2, 9 and 10 (Natural Advantage™, Freehold, New Jersey, U.S.A.), and SUCRAMASK™ (Creative Research Management, Stockton, California, U.S.A.). Suitable polymer additives include, but are not limited to, chitosan, pectin, pectic, pectinic, polyuronic, polygalacturonic acid, starch, food hydrocolloid or crude extracts thereof (e.g., gum acacia Senegal (FIBERGUM™), gum acacia seyal, carageenan), poly-L-lysine (e.g., poly-L-α-lysine or poly-L-ε-lysine), poly-L-ornithine (e.g., poly-L-α-omithine or poly-L-ε-ornithine), polypropylene glycol, polyethylene glycol, poly(ethylene glycol methyl ether), polyarginine, polyaspartic acid, polyglutamic acid, polyethylene imine, alginic acid, sodium alginate, propylene glycol alginate, and sodium polyethyleneglycolalginate, sodium hexametaphosphate and its salts, and other cationic polymers and anionic polymers.

In some example embodiments, the oral composition for hydration or rehydration may comprise protein hydrolysate additive. In related embodiments the composition may include protein hydrolysate additive as a replacement for an ingredient, such as an amino acid.

Suitable surfactant additives include, but are not limited to, polysorbates (e.g., polyoxyethylene sorbitan monooleate (polysorbate 80), polysorbate 20, polysorbate 60), sodium dodecylbenzenesulfonate, dioctyl sulfosuccinate or dioctyl sulfosuccinate sodium, sodium dodecyl sulfate, cetylpyridinium chloride (hexadecylpyridinium chloride), hexadecyltrimethylammonium bromide, sodium cholate, carbamoyl, choline chloride, sodium glycocholate, sodium taurodeoxycholate, lauric arginate, sodium stearoyl lactylate, sodium taurocholate, lecithins, sucrose oleate esters, sucrose stearate esters, sucrose palmitate esters, sucrose laurate esters, and other emulsifiers, and the like.

Suitable flavonoid additives are classified as flavonols, flavones, flavanones, flavan-3-ols, isoflavones, or anthocyanidins. Non-limiting examples of flavonoid additives include, but are not limited to, catechins (e.g., green tea extracts such as Polyphenonm™ 60, Polyphenon™ 30, and Polyphenon™ 25 (Mitsui Norin Co., Ltd., Japan), polyphenols, rutins (e.g., enzyme modified rutin Sanmelin™ AO (San-fi Gen F.F.I., Inc., Osaka, Japan)), neohesperidin, naringin, neohesperidin dihydrochalcone, and the like. Suitable alcohol additives include, but are not limited to, ethanol.

Suitable astringent compound additives include, but are not limited to, tannic acid, europium chloride (EuCl₃), gadolinium chloride (GdCl₃), terbium chloride (TbCl₃), alum, tannic acid, and polyphenols (e.g., tea polyphenols).

### Compositions and Methods

The present composition may generally be in any edible form, such as liquid, semi-liquid, solid, or semi-solid. In some embodiments, the present composition is a beverage or beverage product. One example of the beverage is a ready-to-drink beverage. Ready-to-drink beverages include carbonated and non-carbonated beverages. Carbonated beverages include, but are not limited to, frozen carbonated beverages, enhanced sparkling beverages, cola, fruit-flavored sparkling beverages (e.g. lemon-lime, orange, grape, strawberry and pineapple), ginger-ale, soft drinks and root beer. Non-carbonated beverages include, but are not limited to, fruit juice, fruit-flavored juice, juice drinks, nectars, vegetable juice, vegetable-flavored juice, sports drinks, energy drinks, enhanced water drinks, enhanced water with vitamins, near water drinks (e.g., water with natural or synthetic flavorants), coconut water, tea type drinks (e.g. black tea, green tea, red tea, oolong tea), coffee, cocoa drink, beverage containing milk components (e.g. milk beverages, coffee containing milk components, cafe au lait, milk tea, fruit milk beverages), beverages containing cereal extracts and smoothies.

In a particular embodiment, the present disclosure relates to a sports drink or an enhanced water drink.

The beverage can be a full-calorie beverage that has up to about 120 calories per 8 oz Serving. The beverage can be a mid-calorie beverage that has up to about 60 calories per 8 oz, serving. The beverage can be a low-calorie beverage that has up to about 40 calories per 8 oz. serving. The beverage can be a zero-calorie that has less than about 5 calories per 8 oz. serving.

In another particular embodiment, the beverage does not contain milk and/or dairy components.

In some embodiments, the present beverage has a plasma osmolality in a range from about 250 to about 350 mOsm/kg, or from about 270 to about 330 mOsm/kg, or from about 290 to about 310 mOsm/kg, or from about 290 to about 300 mOsm/kg.

The present disclosure also provides a method of preparing a ready-to-drink beverage comprising (i) providing a beverage matrix and (ii) adding the beverage ingredients described herein above to the beverage matrix, thereby providing a ready-to-drink beverage. The method optionally includes a further mixing step whereby the beverage ingredients and matrix are mixed to promote dissolution. The method can also optionally include a heating step, whereby the beverage ingredients and matrix are heated to promote dissolution.

Beverage ingredients are dissolved in the beverage matrix. Exemplary beverage matrices include water of beverage quality, for example tap water, deionized water, distilled water, reverse osmosis water, carbon-treated water, purified water, demineralized water and combinations thereof. Additional suitable matrices include, but are not limited to phosphoric acid, phosphate buffer, citric acid, citrate buffer and carbon-treated water.

The method can be performed at any temperature required to formulate the ready-to-drink beverage. For example, for ingredients that are temperature sensitive, the method is carried out below 70° C. Similarly, the beverage ingredients can be added to the beverage matrix in any order.

In some embodiments, the present composition is in a dry or semi-dry form such as a dry powder, a beverage concentrate, a tablet, a capsule, a gel, a gum. The dry composition can be readily consumable or be readily and rapidly soluble in a drinkable medium such as water.

In one particular embodiment, the present compositions may be prepared in concentrate or powder form to be reconstituted for use by the subject by the addition of water or any other appropriate liquid. Such reconstitution is made with the requisite amounts of water/liquid to ensure that the beverage to be consumed contains the active components in the proportions previously noted. In another embodiment, the composition may be solubilized in water/liquid and then brought to a frozen state, so as to provide, for example, flavored ices on sticks, like the ones known under the commercial name or trade mark “Popsicle.” In one embodiment, a method for preparing a ready-to-drink solution comprises mixing an oral composition described herein with a drinkable medium in a weight ratio such that the concentration of active ingredients in the formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L. or from about 0.05 g/L to about 1 g/L, based on the total volume of the ready-to-drink solution.

In certain embodiments, the oral composition has a concentration of at least one active ingredient comprising a mixture of sodium salts from about 15 mmol/L and about 35 mmol/L, or about between 17 mmol/L, and about 30 mmol/L, or between about 20 mmol/L and about 30 mmol/L, or between about 23 mmol/L and about 28 mmol/L, or between about 25 mmol/L and about 26 mmol/L, or about 25.9 mmol/L. In some embodiments, the oral composition has a concentration of at least one active ingredient comprising a mixture of potassium salts from about 15 mmol/L and about 35 mmol/L, or about between 17 mmol/L and about 30 mmol/L, or between about 23 mmol/L and about 29 mmol/L, or between about 25 mmol/L and about 28 mmol/L, or about 27.8 mmol/L. In certain embodiments, the at least two active ingredients are a first active ingredient of sodium salts having a concentration between about 10 mmol/L and about 28 mmol/L, or about between 13 mmol/L and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L. Exemplary drinkable media include water of beverage quality, for example tap water, deionized water, distilled water, reverse osmosis water, carbon-treated water, purified water, demineralized water, and combinations thereof. The drinkable medium may also be an existing beverage such as sparkling water, juice, tea, milk, or coffee.

In some embodiments, the present composition may be prepared as a dry powder mixture. The dry powder mixture is combined with a support for dispersion in water/liquid, such as maltodextrin, with a non-glucose sweet taste base, such as neohesperidine dihydrochalcone, at a level so as not to exceed 120 kcal/L, when reconstituted, and possibly flavored with fruit extracts or aromas, such as orange, lemon, strawberry, or others. One dose is intended for dissolution in 591 ml drinking water/liquid (one standard sports bottle). The concentrations are merely indicative, and more concentrated drinks may be prepared on the same formula basis.

The above powder mix may be added to a suitable support for tablet compression, with good organoleptic properties, such as sorbitol and magnesium stearate. The mass is possibly edulcorated with a known natural sweetener such as Neohesperidine dihydrochalcone and the total energy content should not exceed 120 kcal in the average daily intake. The product may be flavored with any choice of fruit or other flavors, such as orange, lemon, menthol, eucaplytol, or the like. The compressed tablets (or equivalent solid forms with the same composition) are intended for an average daily intake of between 5 and 10 tablets.

In some embodiments, the present disclosure relates to methods for fast hydration, fast rehydration, long lasting hydration, and/or long lasting rehydration. The method generally includes administering any oral composition described herein.

The present method generally utilizes the compositions described herein for at least one of the following purposes: providing a quick impact on plasma D0 percent restoration during rehydration stage, attenuating or reversing the effects of dehydration or hypohydration, ameliorating other adverse effects of exercise, heat or other activity which causes bodily fluid loss, providing positive impact on following physical performance, enhancing the duration of body fluid retention, rapidly increasing the plasma D₂O percent, maintaining the increased plasma D₂O percent for long duration, restoring electrolyte balance, providing energy source, improving physical performance, limiting or reducing calorie uptake.

In some embodiments, the present disclosure provides a method of fast hydrating, fast rehydrating, providing long lasting hydration, and/or provide long lasting rehydration to a human, the method comprising administering an effective amount of the oral composition described herein, wherein the oral composition is free from nutritive sweeteners. Administration of the oral composition can be before, during, or after dehydration or loss of body fluid.

In some embodiments, the method comprises administering a sports beverage to an human described herein, wherein the sports drink is further configured to enhance sports performance of the human, reduce lactate production, reduce perceived exhaustion, reduce muscle soreness (both actual muscle damage and perceived soreness), improve time to exhaustion, improve time trial performance, improve power output, reduce production of lactic acid, and reduce net fluid loss observed with exercise. As used herein, “enhanced sports performance” refers to an improvement in sports performance associated with consuming embodiments of the sports beverages provided herein, as compared to sports performance without consuming the sports beverage or water. The sports beverage may be consumed before, during, or after sports performance. As used herein, “sports performance” refers to both endurance exercise and non-endurance exercise. Endurance exercise includes aerobic activities over prolonged periods of time (e.g., greater than about 30 minutes) while non-endurance exercise includes aerobic activities over a shorter period of time (e.g., less than about 30 minutes).

In one particular embodiment, the present disclosure relates to a method of enhancing athletic performance comprising administering/consuming a sports beverage before, during, or after endurance exercise, wherein the sports beverage comprises an aqueous solution described herein, wherein the formulation comprise active ingredients in an amount from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, based on the total volume of the sports beverage. In some embodiments, the oral composition has a concentration of at least one active ingredient comprising a mixture of sodium salts from about mmol/L and about 35 mmol/L, or about between 17 mmol/L and about 30 mmol/L, or between about 20 mmol/L and about 30 mmol/L, or between about 23 mmol/L and about 28 mmol/L, or between about 25 mmol/L and about 26 mmol/L, or about 25.9 mmol/L. In some embodiments, the oral composition has a concentration of at least one active ingredient comprising a mixture of potassium salts from about 15 mmol/L and about 35 mmol/L, or about between 17 mmol/L and about 30 mmol/L, or between about 23 mmol/L and about 29 mmol/L, or between about 25 mmol/L and about 28 mmol/L, or about 27.8 mmol/L. In certain embodiments, the at least two active ingredients are a first active ingredient of sodium salts having a concentration between about 10 mmol/L and about 28 mmol/L, or about between 13 mmol/L and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.

In some embodiments, the method includes preparing a drinkable solution by dissolving a dry powder composition comprising the formulation in a drinkable medium comprising water, and administering the drinkable solution orally. In some embodiments, the concentration of active ingredients in the amino acid-free formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, based on the total volume of the drinkable solution.

In other embodiments, the method includes consuming/ingesting the oral composition and a drinkable medium comprising water. Ingestion of the oral composition and a drinkable medium can be concurrently, simultaneously, separately, or successively. In some embodiments, the ratio of the oral composition to the drinkable medium is such that the content of active ingredients in the formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, relative to the total volume of the drinkable medium. In some embodiments, the oral composition has a concentration of at least one active ingredient comprising a mixture of sodium salts from about mmol/L and about 35 mmol/L, or about between 17 mmol/L and about 30 mmol/L, or between about 20 mmol/L and about 30 mmol/L, or between about 23 mmol/L and about 28 mmol/L, or between about 25 mmol/L and about 26 mmol/L, or about 25.9 mmol/L. In some embodiments, the oral composition has a concentration of at least one active ingredient comprising a mixture of potassium salts from about 15 mmol/L and about 35 mmol/L, or about between 17 mmol/L and about 30 mmol/L, or between about 23 mmol/L and about 29 mmol/L, or between about 25 mmol/L, and about 28 mmol/L, or about 27.8 mmol/L. In certain embodiments, the at least two active ingredients are a first active ingredient of sodium salts having a concentration between about 10 mmol/L and about 28 mmol/L, or about between 13 mmol/L and about 25 mmol/L, or between about 16 mmol/L and about 20 mmol/L, or between about 17 mmol/L. and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.

The methods according to the present disclosure provide fast and effective hydration and retention of body fluid of a human. In some embodiments, the plasma D₂O percent of the human is increased by at least about 2.5%, or at least about 3%, or at least about 4% in about 10 minutes after administration of the oral composition.

In some embodiments, the plasma D₂O percent of the human is increased by at least about 4.5%, or at least about 5%, or at least about 5.5%, or at least about 6% in about 15 minutes after administration of the oral composition.

In some embodiments, the plasma D₂0 percent of the human is increased by at least about 5%, or at least about 5.5%, or at least about 6%, or at least about 6.5%, or at least about 7% in about 30 minutes after administration of the oral composition.

In some embodiments, the plasma D₂O percent of the human is increased by at least about 3%, or at least about 3.5%, or at least about 4%, or at least about 5%, or at least about 6% in about 45 minutes after administration of the oral composition.

In some embodiments, the plasma D₂O percent of the human is increased by at least about 3%, or at least about 3.5%, or at least about 4%, or at least about 5%, or at least about 6% on in about 60 minutes after administration of the oral composition.

In some embodiments, the plasma D₂O percent of the human measured at 30 minutes after administration of the oral composition remains substantially unchanged thereafter for at least about 15 minutes, or at least about 30 minutes, or at least about 1 hour.

In some embodiments, the plasma D₂O percent of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.

In some embodiments of the present method, the plasma osmolality of the human is maintained in a range from about 270 to about 330 mOsm/kg, or from about 280 to about 320 mOsm/kg, or from about 290 to about 310 mOsm/kg, or from about 290 to about 300 mOsm/kg, in at least 60 minutes after administration of the oral composition.

In some embodiments, the change of the plasma osmolality of the human is no greater than 3 mOsm/kg in at least 60 minutes after administration of the oral composition.

### EXAMPLES

### Example 1

A human trial was conducted using example beverages of the present disclosure to measure faster hydration benefit (assessing speed of absorption into the blood stream by measuring plasma deuterium oxide (D₂O) accumulation) and longer lasting hydration benefit (assessing amount of consumed beverage volume by measuring net fluid balance (subtract urinary output from body weight overtime)) (Maughan et al, 2016). This study evaluated the efficacy of 3 test beverages to faster hydration and longer lasting hydration during a 3-hour rehydration period after exercise-induced dehydration in the heat compared to water intake (water as control). Healthy men (n=12) completed 4 dehydration/rehydration trials that differ in the beverages ingested during the rehydration period.

### Method

The study group consisted of 12 apparently healthy men aged 18-40 years, and the study participants were randomly assigned to one of 4 intervention trials (water and 3 test beverages) in a cross-over, double-blinded manner. The test beverages were given an alpha-numeric code in order to maintain a double-blinded study design. All the beverages did not have any nutrients, flavor, nor color, and they were differing only by the electrolyte content of the test beverages.

Each study participant was subjected to a dehydration phase in an environmental chamber capable of maintaining temperature and humidity levels of ~95° F./35° C. and 40% RH (relative humidity) within a range of 2° C. and ±3% RH. Prior to the dehydration phase, a butterfly needle was inserted, and participants sat for 15 minutes before a blood sample was drawn without stasis (Pre-I): pre-dehydration phase). After the fasting blood draw, participants were provided a small snack (Clif Bar, 250-260 kcal, 130-230 mg sodium) to eat in front of study personnel. Immediately before starting the dehydration phase, participants were weighed nude (after Clif bar and blood draw).

During the dehydration phase, participants participated in an intermittent cycling protocol (repeated bouts of 20 min exercise, 5 min rest) at ~95° F., 40% RH to induce body mass loss (dehydration phase). The workload for the cycling exercise was set to a predetermined intensity from the baseline assessment (~50% HRR). During each 5 min rest period, participants were weighed to obtain net fluid loss from baseline.

Following the dehydration phase, participants participated in a rehydration phase. After achieving the target body mass loss, participants were transported back to the laboratory, placed into a temperate environment (~20 to 22° C.), and rested for 15 minutes in a seated position. After 15 minutes of rest, an intravenous catheter was inserted, and participants continued to rest in a seated position for an additional 15 minutes before a pre-rehydration (Pre-R) blood sample was drawn without stasis. Once the Pre-R blood sample was drawn, a total urine sample was collected, and participants were weighed nude to calculate total body mass lost compared to Pre-D levels (% dehydration=% body fluid loss). After this transition resting period (~40 min), participants began the rehydration/recovery phase of the protocol.

During the rehydration phase, participants ingested equal quantities of the assigned beverage (water or test beverages) every 15 min for 60 min (5 total bolus drinks) in order to replace 150% of their body mass lost during the dehydration phase. During the rehydration phase of the protocol, participants were asked to maintain a seated position to limit body fluid shifts. During the recovery phase of the protocol, participants sat quietly for 3 hours in a temperate environment (~20 to 22° C.).

### Faster Hydration (Plasma D₂O) Accumulation)

Beverage appearance in blood was compared across beverages using deuterium oxide dilution according to the technique of Davis et al. (1987). Venous blood samples for D₂O analysis were drawn without stasis from a Teflon indwelling catheter placed before the Rehydration Phase. After maintaining a seated position for at least 20 minutes, a baseline blood sample was obtained immediately before the final drink bolus to control for any background D₂O (3 ml EDTA). Participants then consumed the final bolus of the test beverage in <5 minutes (but timed to be similar across trials) containing D20. A final 50 ml of the beverage was then used as a rinse to ensure all D₂O was ingested. Additional venous blood (3 ml) was obtained at 5-(5 min-R), 10-(10 min-R), 15-(15 min-R), 20-(20 min-R), 25-(25 min-R), 30-(30 min-R), and 45-(45 min-R) minutes following the beginning of rehydration, post-rehydration (Post-R), 20 minutes after the final fluid bolus (20Post-R), 40 minutes after the final fluid bolus (40Post-R), and 60 minutes after the final fluid bolus (60Post-R). Blood samples were centrifuged at 3000 rpm and plasma stored at −80° C. Plasma D₂O enrichment was measured by an independent laboratory. D₂O enrichment in the blood does not provide quantitative measures regarding the amount of fluid emptied from the stomach and absorbed in the intestine but estimates relative differences in volume and rate of delivery (Jeukendrup et al. 2009; Hill et al. 2008).

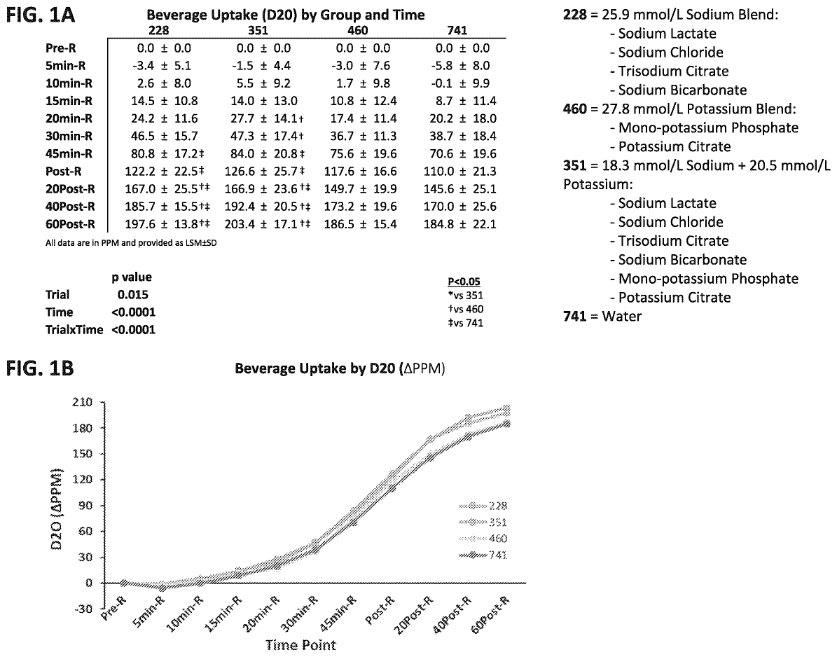

FIG. 1A discloses beverage uptake (D₂O in PPM) by group and time at pre-rehydration (Pre-R), 5-(5 min-R), 10-(10 min-R), 15-(15 min-R), 20-(20 min-R), 25-(25 min-R), 30-(30 min-R), and 45-(45 min-R) minutes following the beginning of rehydration, post-rehydration (Post-R), 20 minutes after the final fluid bolus (20Post-R), 40 minutes after the final fluid bolus (40Post-R), and 60 minutes after the final fluid bolus (60 Post-R) for each of the 3 examples of the present disclosure plus water. Example 228 is a 25.9 mmol/L sodium blend comprising sodium lactate, sodium chloride, trisodium citrate, and sodium bicarbonate. Example 460 is a 27.8 mmol/L potassium blend comprising mono-potassium phosphate and potassium citrate. Example 351 is an 18.3 mmol/L, sodium and 20.5 mmol/L potassium blend comprising sodium lactate, sodium chloride, trisodium citrate, sodium bicarbonate, mono-potassium phosphate and potassium citrate. Example 741 is water. Example 228, example 460, example 351, and example 741 are the same throughout the experiments.

### Longer Lasting Hydration (Net Fluid Balance)

Body weight was measured before dehydration (Pre-D), before rehydration/post dehydration (Post-D), immediately after ingestion of the final fluid bolus (Post-R), and every 60 min after the final fluid bolus (60Post-R, 120Post-R, and 180Post-R). Body mass was measured in duplicate, nude after urinating and toweling dry on an electronic scale accurate to 10 grams. The fluid deficit incurred by the dehydration protocol was calculated from the acute change in body mass from Pre-D to Pre-R (corrected for fecal losses if necessary) and expressed as a percent from baseline body mass (Cheuvront et. al. 2004, 2010). For all calculations, water and body mass losses from sweat and urine were considered equivalent (1 L=1 kg).

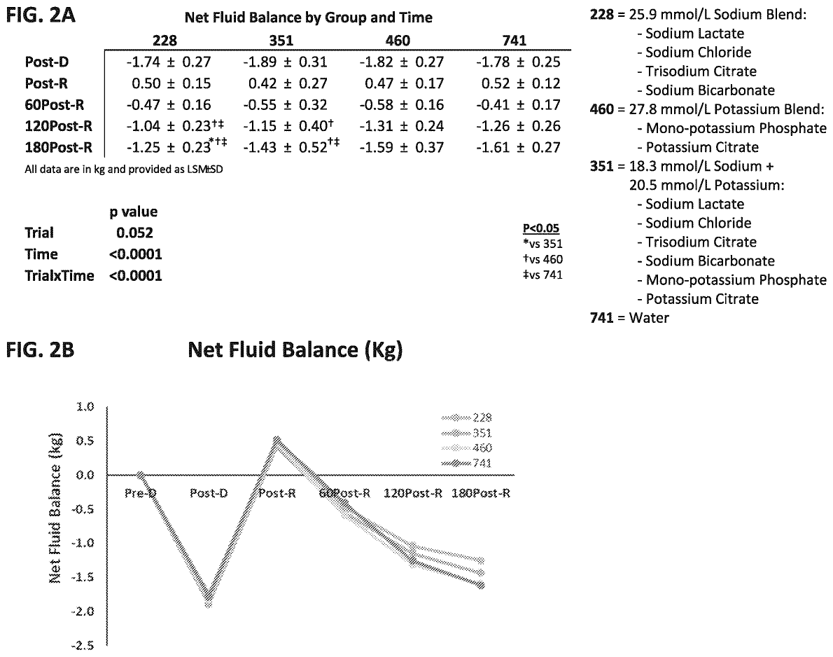

FIG. 2A discloses the net fluid balance (in kgs) by group and time post-dehydration (Post-D), post-rehydration (Post-R), 60 minutes after the final fluid bolus (60Post-R), 120 minutes after the final fluid bolus (120Post-R), and 180 minutes after the final fluid bolus (180Post-R) for each of the 3 examples of the present disclosure plus water.

### Results

### Faster Hydration

FIG. 1B discloses is graph of the data in FIG. 1A. Both the sodium blend (228) and sodium+potassium blend (351) started to have a faster hydration benefit compared to water (741) 45 minutes after the start of rehydration (45 min-R). The sodium blend (228) showed a faster hydration benefit compared to the potassium blend (460) 20 minutes after the final fluid bolus (20Post-R), while sodium+potassium blend (351) showed a faster hydration benefit compared to the potassium blend (460) beginning at 20 minutes after the start of rehydration (20 Min-R).

### Longer Lasting Hydration

FIG. 2B is a graph of the data in FIG. 2A and further includes pre-dehydration (Pre-D) as the zero measurement. The sodium blend (228) showed a longer lasting hydration benefit compared to water at 120- and 180-minutes post-R, while the sodium+potassium blend (351) showed a longer lasting hydration benefit at 180 min post-R compared to water. The sodium blend (228) and sodium+potassium blend (351) demonstrated a longer lasting hydration benefit compared to the potassium blend (460) at 120- and 180-minutes post-R. The sodium blend (228) also showed a longer lasting hydration benefit at 180 minutes post-R compared to the sodium+potassium blend (351).

### NON-PATENT REFERENCES

Siamak, A. Adibi, Leucine absorption rate and net movements of sodium and water in human jejunum, Journal of Applied Physiology, 1970, 28(6), 753-757.
Kazunobu Okazaki, Yoshi-Ichiro Kamijo, Yoshiaki Takeno, Tadashi Okumoto, Shizue Masuki, Hiroshi Nose, Effects of exercise training on thermoregulatory responses and blood volume in older men, Journal of Applied Physiology, 2002, 93, 1630-1637.
Chih-Yin Tai, Jordan M Joy, Paul H Falcone, Laura R Carson, Matt M Mosman, Justen L Straight, Susie L Oury, Carlos Mendez Jr, Nick J Loveridge, Michael P Kim & Jordan R Moon, An amino acid-electrolyte beverage may increase cellular rehydration relative to carbohydrate-electrolyte and flavored water beverages. Nutrition Journal, 2014 (13), 47.
Kurt J Sollanek, Matthew Tsurumoto, Sadasivan Vidyasagar, Robert W Kenefick, Samuel N Cheuvront, Neither body mass nor sex influences beverage hydration index outcomes during randomized trial when comparing 3 commercial beverages. The American Journal of Clinical Nutrition, 2018, 107(4), 544-549.
Samuel N. Cheuvront, Robert W. Kenefick, Nisha Charkoudian. Katherine M. Mitchell, Adam J. Luippold, Karleigh E. Bradbury, Sadasivan Vidyasagar, Efficacy of Glucose or Amino Acid-Based Commercial Beverages in Meeting Oral Rehydration Therapy Goals After Acute Hypertonic and Isotonic Dehydration, J Parenler Enteral Nutr, 2018, 42(7), 1185-1193.
S. M. Shirreffs, A. J. Taylor, J. B. Leiper, R. J. Maughan, Post-exercise rehydration in man: effects of volume consumed and drink sodium content. Medicine and Science in Sports and Exercise, 196, 28(10):1260-1271.
S. N. Cheuvront, R. Carter 3rd, S. J. Montain, M. N. Sawka, Daily body mass variability and stability in active men undergoing exercise-heat stress. Int J Sport Nutr Exerc Metab. 2004, 14(5):532-40.
S N. Cheuvront, B. R. Ely, R. W. Kenefick, M. N. Sawka, Biological variation and diagnostic accuracy of dehydration assessment markers. Am J Clin Nutr. 2010, 92(3):565-73.
L. B. Baker, Sweating Rate and Sweat Sodium Concentration in Athletes: A Review of Methodology and Intra/Interindividual Variability. Sports Med. 2017, 47(Suppl 1):111-128.

All publications, patents and patent applications mentioned in the specification are indicative of the level of those skilled in the art to which this disclosure pertains.

All of the compositions and methods disclosed and claimed herein can be made and executed without undue experimentation in light of the present disclosure. While the compositions and methods of this disclosure have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the disclosure. More specifically, it will be apparent that certain agents, additives, and ingredients that are similar according to their physical, chemical, physiological, and/or gustative properties may be substituted for the agents, additives and ingredients described herein while the same or similar results would be achieved. All such similar substitutes and modifications apparent to those skilled in the art are deemed to be within the spirit, scope, and concept of the disclosure as defined by the hereinafter appended claims.

### CLAUSES

1. An oral composition for fast hydration, fast rehydration, long lasting hydration and/or long lasting rehydration, the oral composition comprising at least one active ingredient.
2. The oral composition of clause 1, wherein the oral composition is an amino acid free formulation and/or substantially free of nutritive sweetener. 3 The oral composition of any one of clauses 1-2, further comprising at least two active ingredients.
4. The oral composition of any one of clauses 2-3, wherein the at least one active ingredient comprises at least one electrolyte selected from the group consisting of sodium, potassium, calcium, magnesium, chloride, phosphate, bicarbonate, and combinations thereof, or wherein the at least two active ingredients comprise at least two electrolytes selected from the group consisting of sodium, potassium, calcium, magnesium, chloride, phosphate, bicarbonate, and combinations thereof.
5. The oral composition of clause 4, wherein the at least one active ingredient comprises two or more electrolytes selected from the group consisting of sodium, potassium, calcium, magnesium, chloride, phosphate, bicarbonate, and combinations thereof, or three or more electrolytes selected from the group consisting of sodium, potassium, calcium, magnesium, chloride, phosphate, bicarbonate, and combinations thereof, or four or more electrolytes selected from the group consisting of sodium, potassium, calcium, magnesium, chloride, phosphate, bicarbonate, and combinations thereof, or five or more electrolytes selected from the group consisting of sodium, potassium, calcium, magnesium, chloride, phosphate, bicarbonate, and combinations thereof, or 6 or more electrolytes selected from the group consisting of sodium, potassium, calcium, magnesium, chloride, phosphate, bicarbonate, and combinations thereof.
6. The oral composition of clause 4, wherein the at least two active ingredient comprises two or more electrolytes selected from the group consisting of sodium, potassium, calcium, magnesium, chloride, phosphate, bicarbonate, and combinations thereof, or three or more electrolytes selected from the group consisting of sodium, potassium, calcium, magnesium, chloride, phosphate, bicarbonate, and combinations thereof, or four or more electrolytes selected from the group consisting of sodium, potassium, calcium, magnesium, chloride, phosphate, bicarbonate, and combinations thereof, or five or more electrolytes selected from the group consisting of sodium, potassium, calcium, magnesium, chloride, phosphate, bicarbonate, and combinations thereof, or 6 or more electrolytes selected from the group consisting of sodium, potassium, calcium, magnesium, chloride, phosphate, bicarbonate, and combinations thereof
7. The oral composition of any one of clauses 2-6, wherein the at least one active ingredient is a mixture of sodium salts.
8. The oral composition of any one of clauses 2-6, wherein the at least one active ingredient is a mixture of potassium salts.
9. The oral composition of any one of clauses 3-6, wherein the at least two active ingredients is a mixture of sodium salts and potassium salts.
10. The oral composition of any one of clauses 3-7, wherein the at least one active ingredient is a mixture of sodium salts comprising sodium lactate, sodium chloride, trisodium citrate, and sodium bicarbonate.
11. The oral composition of any one of clause 3-6, or 8, wherein the at least one active ingredient is a mixture of potassium salts comprising mono-potassium phosphate and potassium citrate.
12. The oral composition of clause 9, wherein the at least two active ingredients is mixture of sodium salts and potassium salts comprising sodium lactate, sodium chloride, trisodium citrate, sodium bicarbonate, mono-potassium phosphate and potassium citrate.
13. The oral composition of any one of clauses 1-12, wherein the at least one active ingredient is a mixture of sodium salts and has a concentration between about 15 mmol/L and about 35 mmol/L, or about between 17 mmol/L and about 30 mmol/L, or between about 20 mmol/L and about 30 mmol/L, or between about 23 mmol/L and about 28 mmol/L, or between about 25 mmol/L and about 26 mmol/L, or about 25.9 mmol/L.
14. The oral composition of any one of clauses 1-12, wherein the at least one active ingredient is a mixture of potassium salts and has a concentration between about mmol/L and about 35 mmol/L, or about between 17 mmol/L and about 30 mmol/L, or between about 23 mmol/L and about 29 mmol/L, or between about 25 mmol/L and about 28 mmol/L, or about 27.8 mmol/L.
15. The oral composition of any one of clauses 1-12, wherein the at least two active ingredients are a first active ingredient of sodium salts having a concentration between about 10 mmol/L and about 28 mmol/L, or about between 13 mmol/L. and about 25 mmol/L, or between about 16 mmol/L and about 20 mmol/L, or between about 17 mmol/L and about 19 mmol/L, or about 18.3 mmol/L. and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.
16. The oral composition of any one of clauses 2-15, wherein the total electrolyte concentration is at least about 200 mg/L.
17. The oral composition of any one of clauses 1-16, further comprising at least one sweetener.
18. The oral composition of clause 17, wherein the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin 1, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, nectame, advantame, glucosylated steviol glycosides (GSGs), and combinations thereof.
19. The oral composition of any one of clauses 1-18, further comprising at least one additive.
20. The oral composition of any one of clauses 1-18, further comprising at least one functional ingredient.
21. The oral composition of any one of clauses 1-20, wherein the oral composition is selected from a sports drink and an enhanced water drink.
22. The oral composition of any one of clauses 1-21, wherein the oral composition is selected from a full-calorie beverage, a mid-calorie beverage, a low-calorie beverage, or a zero-calorie beverage.
23. The oral composition of any one of clauses 1-22, wherein the oral composition is a ready-to-drink hydrating beverage.
24. The oral composition of any one of clauses 1-22, wherein the oral composition is in a dry or semi-dry form.
25. The oral composition of clause 24, wherein the oral composition is a gum, tablet, capsule, or dry powder.
26. A method of fast hydrating, fast rehydrating, providing long lasting hydration, and/or providing long lasting rehydration in a human, the method comprising administering an oral composition, the oral composition comprising at least one active ingredient.
27. The method of clause 26, wherein the human obtains a fast hydration, fast rehydration, long lasting hydration, and/or long lasting rehydration from the administration of the oral composition.
28. The method of any of clauses 26-27, wherein the oral composition is a ready-to-drink hydrating beverage, or a sports drink, or an enhanced water drink.
29. The method of any one of clauses 26-28, wherein the oral composition is selected from a full-calorie beverage, a mid-calorie beverage, a low-calorie beverage, or a zero-calorie beverage.
30. The method of any one of clauses 26-29, wherein the oral composition is an amino acid free formulation and/or substantially free of nutritive sweetener.
31. The method of any one of clauses 26-29, wherein the oral composition further comprises at least two active ingredients.
32. The method of any one of clauses 30-31, wherein the at least one active ingredient comprises at least one electrolyte selected from the group consisting of sodium, potassium, calcium, magnesium, chloride, phosphate, bicarbonate, and combinations thereof, or wherein the at least two active ingredients comprise at least two electrolytes selected from the group consisting of sodium, potassium, calcium, magnesium, chloride, phosphate, bicarbonate, and combinations thereof.
33. The method of clause 32, wherein the at least one active ingredient comprises two or more electrolytes selected from the group consisting of sodium, potassium, calcium, magnesium, chloride, phosphate, bicarbonate, and combinations thereof, or three or more electrolytes selected from the group consisting of sodium, potassium, calcium, magnesium, chloride, phosphate, bicarbonate, and combinations thereof, or four or more electrolytes selected from the group consisting of sodium, potassium, calcium, magnesium, chloride, phosphate, bicarbonate, and combinations thereof, or five or more electrolytes selected from the group consisting of sodium, potassium, calcium, magnesium, chloride, phosphate, bicarbonate, and combinations thereof, or 6 or more electrolytes selected from the group consisting of sodium, potassium, calcium, magnesium, chloride, phosphate, bicarbonate, and combinations thereof.
34. The method of clause 32, wherein the at least two active ingredient comprises two or more electrolytes selected from the group consisting of sodium, potassium, calcium, magnesium, chloride, phosphate, bicarbonate, and combinations thereof, or three or more electrolytes selected from the group consisting of sodium, potassium, calcium, magnesium, chloride, phosphate, bicarbonate, and combinations thereof, or four or more electrolytes selected from the group consisting of sodium, potassium, calcium, magnesium, chloride, phosphate, bicarbonate, and combinations thereof, or five or more electrolytes selected from the group consisting of sodium, potassium, calcium, magnesium, chloride, phosphate, bicarbonate, and combinations thereof, or 6 or more electrolytes selected from the group consisting of sodium, potassium, calcium, magnesium, chloride, phosphate, bicarbonate, and combinations thereof.
35. The method of any one of clauses 27-34, wherein the at least one active ingredient is a mixture of sodium salts.
36. The method of any one of clauses 27-34, wherein the at least one active ingredient is a mixture of potassium salts.
37. The method of any one of clauses 27-34, wherein the at least two active ingredients is a mixture of sodium salts and potassium salts.
38. The method of any one of clauses 27-34, wherein the at least one active ingredient is a mixture of sodium salts comprising sodium lactate, sodium chloride, trisodium citrate, and sodium bicarbonate.
39. The method of any one of clauses 27-34, or 36, wherein the at least one active ingredient is a mixture of potassium salts comprising mono-potassium phosphate and potassium citrate.
40. The method of clause 37, wherein the at least two active ingredients is mixture of sodium salts and potassium salts comprising sodium lactate, sodium chloride, trisodium citrate, sodium bicarbonate, mono-potassium phosphate and potassium citrate.
41. The method of any one of clauses 26-40, wherein the at least one active ingredient is a mixture of sodium salts and has a concentration between about 15 mmol/L and about 35 mmol/L, or about between 17 mmol/L and about 30 mmol/L, or between about 20 mmol/L and about 30 mmol/L, or between about 23 mmol/L and about 28 mmol/L, or about 25 mmol/L, between about 25 mmol/L and about 26 mmol/L, or about 25.9 mmol/L.
42. The method of any one of clauses 26-40, wherein the at least one active ingredient is a mixture of potassium salts and has a concentration between about mmol/L and about 35 mmol/L, or about between 17 mmol/L and about 30 mmol/L, or between about 20 mmol/L and about 29 mmol/L, or between about 25 mmol/L and about 28 mmol/L, or about 27.8 mmol/L.
43. The method of any one of clauses 26-40, wherein the at least two active ingredients are a first active ingredient of sodium salts having a concentration between about 10 mmol/L and about 28 mmol/L, or about between 13 mmol/L and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/IL and about 21 mmol/L, or about 20.5 mmol/L.
44. The method of any one of clauses 27-43, wherein the total electrolyte concentration is at least about 200 mg/L.
45. The method of any one of clauses 26-44, wherein the oral composition further comprises at least one sweetener.
46. The method of clause 45, wherein the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs), and combinations thereof.
47. The method of any one of clauses 26-46, wherein the oral composition further comprises at least one additive.
48. The method of any one of clauses 26-47, wherein the oral composition further comprises at least one functional ingredient.
49. The method of any one of clauses 26.48, wherein the oral composition is in a dry or semi-dry form.
50. The method of clause 49, wherein the oral composition is a gum, tablet, capsule, or dry powder.
51. The method of any one of clauses 26-50, wherein the plasma D₂O percent of the human is increased by at least about 2.5%, or at least about 3%, or at least about 4% on or before 5 minutes after administration of the oral composition.
52. The method of any one of clauses 26-50, wherein the plasma D0 percent of the human is increased by at least about 4.5%, or at least about 5%, or at least about 5.5%, or at least about 6% on or before 15 minutes after administration of the oral composition.
53. The method of any one of clauses 26-50, wherein the plasma D₂O percent of the human is increased by at least about 5%, or at least about 5.5%, or at least about 6^{%}, or at least about 6.5%, or at least about 7% on or before 30 minutes after administration of the oral composition.
54. The method of any one of clauses 26-50, wherein the plasma D₂O percent of the human measured at 30 minutes after administration of the oral composition remains substantially unchanged thereafter for at least about 15 minutes, or at least about 30 minutes, or at least about 1 hour.
55. The method of any one of clauses 26-54, wherein the plasma D₂O percent of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.
56. The method of any one of clauses 26-54, wherein plasma osmolality of the human is maintained in a range from about 270 to about 330 mOsm/kg, or from about 280 to about 320 mOsm/kg, or from about 290 to about 310 mOsm/kg, or from about 290 to about 300 mOsm/kg, in at least 60 minutes after administration of the oral composition.
57. The method of any one of clauses 26-56, wherein plasma osmolality of the human prior to administration of the oral composition is changed by no greater than 3 mOsm/kg after at least 60 minutes after administration of the oral composition.
58. A method for improving physical or sports performance, the method comprising administering any oral composition according to clauses 1-25.
59. The method of clause 34, wherein the oral composition is administered before, during, or after endurance exercise.
60. The method of any of clauses 34-35, wherein the enhanced performance is characterized by reduced perceived physical exhaustion, reduced muscle soreness, reduced muscle damage, reduced net fluid loss, reduced production of lactic acid, improved time to exhaustion, improved time trial performance, improved power output or any combinations thereof.

What is claimed is:

1. An oral composition for fast hydration, fast rehydration, long lasting hydration, and/or long lasting rehydration, the oral composition comprising at least one active ingredient.

2. The oral composition of claim 1, wherein the oral composition is an amino acid free formulation and/or substantially free of nutritive sweetener.

3. The oral composition of claim 1, further comprising at least two active ingredients.

4. The oral composition of claim 3, wherein the at least two active ingredients comprise at least two electrolytes selected from the group consisting of sodium, potassium, calcium, magnesium, chloride, phosphate, bicarbonate, and combinations thereof.

5. The oral composition of claim 1, wherein the at least one active ingredient is a mixture of sodium salts.

6. The oral composition of claim 1, wherein the at least one active ingredient is a mixture of potassium salts.

7. The oral composition of claim 3, wherein the at least two active ingredients is a mixture of sodium salts and potassium salts.

8. The oral composition of claim 5, wherein the at least one active ingredient is a mixture of sodium salts comprising sodium lactate, sodium chloride, trisodium citrate, and sodium bicarbonate.

9. The oral composition of claim 6, wherein the at least one active ingredient is a mixture of potassium salts comprising mono-potassium phosphate and potassium citrate.

10. The oral composition of claim 7, wherein the at least two active ingredients is a mixture of sodium salts and potassium salts comprising sodium lactate, sodium chloride, trisodium citrate, sodium bicarbonate, mono-potassium phosphate and potassium citrate.

11. The oral composition of claim 8, wherein the at least one active ingredient is a mixture of sodium salts and has a concentration between about 15 mmol/L and about 35 mmol/L, or about between 17 mmol/L, and about 30 mmol/L, or between about 20 mmol/L and about 30 mmol/L, or between about 23 mmol/L and about 28 mmol/L, or between about 25 mmol/L and about 26 mmol/L, or about 25.9 mmol/L.

12. The oral composition of claim 9, wherein the at least one active ingredient is a mixture of potassium salts and has a concentration between about 15 mmol/L and about mmol/L, or about between 17 mmol/L and about 30 mmol/L, or between about 23 mmol/L and about 29 mmol/L, or between about 25 mmol/L and about 28 mmol/L, or about 27.8 mmol/L.

13. The oral composition of claim 10, wherein the at least two active ingredients are a first active ingredient of sodium salts having a concentration between about mmol/L and about 28 mmol/L, or about between 13 mmol/L and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.

14. The oral composition of claim 13, wherein the total electrolyte concentration is at least about 200 mg/L.

15. The oral composition of claim 13, further comprising at least one sweetener.

16. The oral composition of claim 15, wherein the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs), and combinations thereof.

17. The oral composition of claim 15, further comprising at least one additive.

18. The oral composition of claim 17, further comprising at least one functional ingredient.

19. The oral composition of claim 18, wherein the oral composition is selected from a sports drink and an enhanced water drink.

20. The oral composition of claim 17, wherein the oral composition is selected from a fill-calorie beverage, a mid-calorie beverage, a low-calorie beverage, or a zero-calorie beverage.

21. The oral composition of claim 19, wherein the oral composition is a ready-to-drink hydrating beverage.

22. The oral composition of claim 19, wherein the oral composition is in a dry or semi-dry form.

23. The oral composition of claim 22, wherein the oral composition is a gum, tablet, capsule, or dry powder.

24. A method of fast hydrating, fast rehydrating, long lasting hydration, and/or long lasting rehydration in a human, the method comprising administering an oral composition, the oral composition comprising at least one active ingredient.

25. The method of claim 24, wherein the human obtains a fast hydration, fast rehydration, long lasting hydration, and/or long lasting rehydration from the administration of the oral composition.

26. The method of claim 25, wherein the oral composition is a ready-to-drink hydrating beverage, or a sports drink, or an enhanced water drink.

27. The method of claim 25, wherein the oral composition is selected from a full-caloric beverage, a mid-calorie beverage, a low-calorie beverage, or a zero-calorie beverage.

28. The method of claim 26, wherein the oral composition is an amino acid free formulation and/or substantially free of nutritive sweetener.

29. The method of claim 26, wherein the oral composition further comprises at least two active ingredients.

30. The method of claim 29, wherein the at least two active ingredients comprise at least one two electrolytes selected from the group consisting of sodium, potassium, calcium, magnesium, chloride, phosphate, bicarbonate, and combinations thereof.

31. The method of claim 26, wherein the at least one active ingredient is a mixture of sodium salts.

32. The method of claim 26, wherein the at least one active ingredient is a mixture of potassium salts.

33. The method of claim 29, wherein the at least two active ingredients is a mixture of sodium salts and potassium salts.

34. The method of claim 31, wherein the at least one active ingredient is a mixture of sodium salts comprising sodium lactate, sodium chloride, trisodium citrate, and sodium bicarbonate.

35. The method of claim 32, wherein the at least one active ingredient is a mixture of potassium salts comprising mono-potassium phosphate and potassium citrate.

36. The method of claim 33, wherein the at least two active ingredients is a mixture of sodium salts and potassium salts comprising sodium lactate, sodium chloride, trisodium citrate, sodium bicarbonate, mono-potassium phosphate and potassium citrate.

37. The method of claim 34, wherein the at least one active ingredient is a mixture of sodium salts and has a concentration between about 15 mmol/L and about 35 mmol/L, or about between 17 mmol/L and about 30 mmol/L, or between about 20 mmol/L and about 30 mmol/L, or between about 23 mmol/L and about 28 mmol/L, or between about 25 mmol/L and about 26 mmol/L, or about 25.9 mmol/L.

38. The method of claim 35, wherein the at least one active ingredient is a mixture of potassium salts and has a concentration between about 15 mmol/L and about 35 mmol/L, or about between 17 mmol/L and about 30 mmol/L, or between about 23 mmol/L and about 29 mmol/L, or between about 25 mmol/L and about 28 mmol/L, or about 27.8 mmol/L.

39. The method of claim 36, wherein the at least two active ingredients are a first active ingredient of sodium salts having a concentration between about 10 mmol/L and about 28 mmol/L, or about between 13 mmol/L and about 25 mmol/L, or between about 16 mmol/L and about 20 mmol/L, or between about 17 mmol/L and about 19 mmol/L, or about 18.3 mmol/L and a second active ingredient of potassium salts having a concentration between about 10 mmol/L and about 28 mmol/L, or about between 13 mmol/L and about 25 mmol/L, or between about 16 mmol/L and about 22 mmol/L, or between about 18 mmol/L and about 21 mmol/L, or about 20.5 mmol/L.

40. The method of claim 39, wherein the total electrolyte concentration is at least about 200 mg/L.

41. The method of claim 39, wherein the oral composition further comprises at least one sweetener.

42. The method of claim 41, wherein the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin 1, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs), and combinations thereof.

43. The method of claim 41, wherein the oral composition further comprises at least one additive.

44. The method of claim 43, wherein the oral composition further comprises at least one functional ingredient.

45. The method of claim 44, wherein the oral composition is in a dry or semi-dry form.

46. The method of claim 45, wherein the oral composition is a gum, tablet, capsule, or dry powder.

47. The method of claim 46, wherein the plasma D₂O percent of the human is increased by at least about 2.5%, or at least about 3%, or at least about 4% on or before 5 minutes after administration of the oral composition.

48. The method of claim 46, wherein the plasma D₂O percent of the human is increased by at least about 4.5%, or at least about 5%, or at least about 5.5%, or at least about 6% on or before 15 minutes after administration of the oral composition.

49. The method of claim 46, wherein the plasma DO percent of the human is increased by at least about 5%, or at least about 5.5%, or at least about 6%, or at least about 6.5%, or at least about 7% on or before 30 minutes after administration of the oral composition.

50. The method of claim 46, wherein the plasma D₂O percent of the human measured at 30 minutes after administration of the oral composition remains substantially unchanged thereafter for at least about 15 minutes, or at least about 30 minutes, or at least about 1 hour.

51. The method of claim 46, wherein the plasma D₂0 percent of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.

52. The method of claim 46, wherein plasma osmolality of the human is maintained in a range from about 270 to about 330 mOsm/kg, or from about 280 to about 320 mOsm/kg, or from about 290 to about 310 mOsm/kg, or from about 290 to about 300 mOsm/kg, in at least 60 minutes after administration of the oral composition.

53. The method of claim 46, wherein plasma osmolality of the human prior to administration of the oral composition is changed by no greater than 3 mOsm/kg after at least 60 minutes after administration of the oral composition.

54. A method for improving physical or sports performance, the method comprising administering any oral composition according to claim 13.

55. The method of claim 54, wherein the oral composition is administered before, during, or after endurance exercise.

56. The method of claim 55, wherein the enhanced performance is characterized by reduced perceived physical exhaustion, reduced muscle soreness, reduced muscle damage, reduced net fluid loss, reduced production of lactic acid, improved time to exhaustion, improved time trial performance, improved power output or any combinations thereof.
